(19) United States
(12) Patent Application Publication
(10) Pub. No.: US 2024/0033066 A1
(43) Pub. Date: Feb. 1, 2024

(54) SYSTEMS AND METHODS FOR MINIMIZING FIBROTIC SCAR FORMATION SUBSEQUENT TO TRAUMA IN TUBULAR ORGANS

(71) Applicant: Harvard Apparatus Regenerative Technology, Inc., Holliston, MA (US)

(72) Inventors: William Fodor, Holliston, MA (US); Linghui Meng, Framingham, MA (US)

(21) Appl. No.: 18/228,828

(22) Filed: Aug. 1, 2023

Related U.S. Application Data
(60) Provisional application No. 63/394,266, filed on Aug. 1, 2022.

(51) Int. Cl. A61F 2/04; A61L 27/18; A61L 27/38

(52) U.S. Cl. CPC A61F 2/04; A61L 27/18; A61L 27/3834; A61L 27/3882; A61F 2002/044; A61F 2002/045

(57) ABSTRACT

Aspects of the disclosure relate methods and synthetic structural supports for regenerating gastrointestinal tissue (e.g., esophageal tissue).

The present disclosure is a non-provisional application that claims the benefit of priority to U.S. Provisional Patent Application 63/394,266 filed Aug. 1, 2022, the specification of which is incorporated by reference herein.

### TECHNICAL FIELD

This disclosure relates to engineered tissues that are useful for replacement or repair of damage tissues. More particularly, the present disclosure pertains to the reduction or elimination of stricture formation subsequent to trauma in tubular organs.

### BACKGROUND

Engineered biological tissues that are useful for replacement or repair of damaged tissues are often produced by seeding cells on synthetic structural supports and exposing the cells to conditions that permit them to synthesize and secrete extracellular matrix components on the structural support. Different techniques have been used for producing synthetic structural supports, including nanofiber assembly, casting, printing, physical spraying (e.g., using pumps and syringes), electrospinning, electrospraying and other techniques for depositing one or more natural or synthetic polymers or fibers to form a structural support having a suitable shape and size for transplanting into a subject (e.g., a human subject, for example, in need of an organ or region of engineered tissue).

It is estimated that over 500.000 individuals worldwide are diagnosed with esophageal malignancy each year. Congenital malformations of the esophagus, such as esophageal atresia, have an average prevalence of 2.44 per 10,000 births. Chronic esophageal stricture after esophageal injury is also common. While there have been advances in minimization of the extent of esophageal resection for early-stage malignant disease, such as endoscopic mucosal resection, the mainstay of treatment for many esophageal disorders is surgical esophagectomy. Traditionally, autologous conduits such as stomach, small bowel, or colon are harvested and rerouted into the chest to restore gastrointestinal continuity. Many children with esophageal atresia or patients affected by either trauma or caustic injury to the esophagus ultimately undergo similar reconstruction. These treatment modalities are associated with high morbidity and mortality.

Autologous conduits are traditionally used because of the complex structure of the esophagus. Comprised of stratified squamous epithelium, submucosa and outer circular and longitudinal muscle layers, these multiple layers of the esophagus provide a barrier to contain oral intake and contamination from escape outside of the gastrointestinal tract. Furthermore, the combined layers provide a physiological mechanism for propulsion, and management of stresses during passage of the bolus either during swallowing or emesis.

Various situations can give rise to damage that can ultimately result in stricture formation in tubular organs. Such situations can include injury, disease, as well as congenital defects that produce damage to tubular organs that can either result in stricture formation directly or necessitate surgical intervention to restore organ integrity and continuity that results in stricture formation as a surgical side effect.

It would be desirable to provide structure as well as a method of making a structure that can support tissue regeneration. It would also be desirable to provide a structure as well as a method of making a structure that would minimize or eliminate fibrotic scar formation in a tubular organ such as a gastrointestinal organ (e.g. esophagus, stomach, etc.) Also disclosed is the use a cellular delivery device for use in reparative or surgical procedures to promote the ordered production of tubular tissue during healing process with a reduces or minimized production of fibrotic scar formation.

### SUMMARY

Disclosed herein is the use of a cellular delivery device and support structure in procedures and therapies for the reduction of strictures in hollow tubular organs. Also disclosed is the use of a cellular delivery device in procedures involving connection of the hollow tubular organ to another organ. Also disclosed are methods for performing surgical and post-surgical procedures in hollow tubular organs such as the esophagus that can reduce or eliminate structural formation in the organ in question. Certain implementations that pertain to synthetic structural supports and related systems that enable production of gastrointestinal tissues (e.g., tissues of the esophagus, stomach, intestine, colon, or other hollow gastrointestinal tissue). In some embodiments, structural supports provide guides for gastrointestinal (e.g., esophageal) tissue growth and regeneration in a subject. In some embodiments, the regenerated gastrointestinal tissue comprises muscle tissue, nervous system tissue, or muscle tissue and nervous system tissue.

In some embodiments, gastrointestinal (e.g., esophageal) tissue is regenerated surrounding a structural support. In some embodiments, the structural support is not incorporated into the final regenerated tissue (e.g., the new esophageal tissue does not incorporate the structural support into the regenerated esophageal walls). Accordingly, aspects of the disclosure relate to guided tissue regeneration where a structural support provides support and/or signals that promote host tissue regeneration without the structural support needing to be incorporated into the regenerated tissue (e.g., without the structural support providing structural or functional support in the final regenerated tissue). Certain implementations can also include procedures where the reduction or minimization of fibrotic scar tissue is desirable such as areas prone to stricture formation.

In some embodiments, a gastrointestinal (e.g., esophageal) structural support includes biodegradable and/or bioresorbable material that is resorbed after gastrointestinal (e.g., esophageal) tissue regeneration is initiated (e.g., after functional esophageal tissue is regenerated).

In some embodiments, a gastrointestinal (e.g., esophageal) structural support includes one or more structures that can be used to assist in removing the structural support after gastrointestinal (e.g., esophageal) tissue regeneration is initiated (e.g., after functional esophageal tissue is regenerated).

In some embodiments, a structural support is cellularized with one or more cell types prior to implantation. In some embodiments, the cells are autologous cells. In some embodiments, the cells are progenitor or stems cells. In some embodiments, the cells are obtained from bone marrow, adipogenic tissue, esophageal tissue, or other suitable tissue. In some embodiments, the cells can be obtained from various allogenic sources, including but not limited to sources such as amniotic fluid, cord bold and the like. In some embodiments, the cells are mesenchymal stem cells (MSCs)

In some embodiments, a structural support is implanted at a site that provides a sufficient stem cell niche (e.g., an esophageal or other gastrointestinal site that provides a stem cell niche) for regenerating tissue in the subject. As used herein, the term “stem cell niche” is defined as a microenvironment which interacts with stem cells to regulate the fate of stem cells, those introduced with the structural support and/or those present in the patient. In some embodiments, without wishing to be bound by theory, the structural support member and/or cells that are provided on the structural support help promote growth and/or regeneration of gastrointestinal tissue from host stem cells present at the site of structural support member implantation.

In some aspects, the disclosure relates to the discovery that growth of esophageal tissues can be promoted or enhanced by the presence of synthetic structural supports that are engineered to replace or repair natural structural patterns and/or functional properties of diseased or injured tissues or organs, without the structural support member s becoming fully integrated into the final regenerated tissue. Thus, in some aspects, the disclosure provides a method for promoting or enhancing growth of gastrointestinal (e.g., esophageal) tissue, the method comprising: delivering to a gastrointestinal (e.g., esophageal) region of a subject a synthetic structural support member, wherein delivery of the synthetic structural support results in growth of new gastrointestinal (e.g., esophageal) tissue in that region of the subject. In some embodiments, the diseased or injured gastrointestinal region is removed (e.g., surgically) prior to implanting the structural support member. In some embodiments, the structural support is an approximately tubular structure that is implanted (e.g., sutured to the ends of the remaining gastrointestinal tissue after removal of the diseased or damaged tissue). In some embodiments, the implanted structural support is shorter than the tissue that was removed (e.g., 5-50% shorter). In some embodiments, the remaining gastrointestinal tissue near the site of the implant is stretched when the tissue is attached (e.g., sutured) to both ends of the structural support. In some embodiments, new gastrointestinal (e.g., esophageal) tissue is regenerated over the implanted structural support without being fully integrated with the structural support. In some embodiments, the walls of the regenerated tissue do not incorporate the walls of the structural support even though the structural support member can be retained within the lumen of the regenerated tissue. In some embodiments, the structural support member can be removed from the lumen formed by the regenerated tissue at a suitable point in the tissue regeneration process.

In some embodiments, the growth of new gastrointestinal (e.g., esophageal) tissue results in the formation of functional tissue (e.g., a functional esophagus) that does not require the continued presence of the structural support for function.

In some embodiments, the synthetic structural support member is resorbable or dissolvable under physiological conditions. In some embodiments, the synthetic structural support member is removed from the gastrointestinal (e.g., esophageal) region of the subject after the formation of a functional esophagus.

In some embodiments, methods and compositions described herein also can be used for tracheal and/or bronchial tissue regeneration.

In certain embodiments, the synthetic structural support member is used in a procedure that involves the joining of two discrete gastrointestinal tissues to one another either in direct contact to one another each other or in position the synthetic structural support member in bridged relationship between the respective regions. In certain embodiments, the two discrete gastrointestinal tissues can be the esophagus and the stomach; the stomach and the small intestine; the small intestine and the large intestine. In certain embodiments, the two discrete regions of the tubular gastrointestinal tissue can be the esophagus and the stomach as would occur in procedures such as a gastric pull-up procedure.

These and other aspects are described in more detail herein.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a perspective view of an embodiment of a synthetic structural support member as disclosed herein with a portion being rendered in partial cross-section;

FIG. 1B is a photomicrograph of a surface of a tubing surface of an embodiment of the synthetic structural support member as disclosed herein;

FIG. 1C side perspective view of a second embodiment of a synthetic structural support member as disclosed herein.

FIG. 2 is a non-limiting depiction of the biological layers of an esophagus; and

FIG. 3 illustrates a non-limiting example of regenerated esophageal tissue in comparison with corresponding native tissue;

FIG. 4 A is a SEM photomicrograph of an outer surface region of an embodiment of the synthetic structural support member as disclosed herein showing cellular growth after seven days of bioreaction taken at 5000×;

FIG. 4B is a photomicrograph of an outer surface region of an embodiment of the synthetic structural support member as disclosed herein showing cellular growth after seven days of bioreaction;

FIG. 5 is a process diagram of a first embodiment of the regeneration method as disclosed herein;

FIG. 6 is an overall study flow for an embodiment of the process as disclosed herein including generation of a cellularized structural support member and subsequent implantation

FIG. 7A are SEMs of samples of an electrospun structural support member according to an embodiment as disclosed herein taken at 1000×, 2000× and 5000× respectively;

FIG. 7B is a graphic depiction of representative uniaxial mechanical testing loading of pre-implantation and post-implantation electrospun structural support member s according to an embodiment as disclosed herein;

FIG. 7C is a Table directed to the uniaxial mechanical properties of pre- and post-implantations structural support members prepared according to an embodiment as disclosed herein;

FIG. 8 is a diagrammatic representation of flow cytometry of MSCs isolated and propagated from adipose tissue for up to 5 passages;

FIG. 9 is an overview of implantation surgery according to an embodiment as disclosed herein;

FIG. 10 is a representation of a timeline according to an embodiment of the process as disclosed herein;

FIG. 11A is a photograph of regenerated esophageal tissue that is located on the interior of the esophagus of a first test subject an esophageal resection site of the first test subject at defined intervals taken after removal of an embodiment of the structural support member device as disclosed herein at 3 to 4 weeks post-surgery;

FIG. 11B is a photograph of regenerated tissue that is located on the interior of the esophagus at the esophageal resection site of FIG. 11A at an intermediate interval after removal of the structural support member device showing tissue growth;

FIG. 11C is a photograph of regenerated tissue that is located on the interior of the esophagus at the esophageal resection site of FIG. 11A at an interval subsequent to the intermediate interval of FIG. 11B showing tissue growth;

FIG. 12A is a photograph of regenerated esophageal tissue that is located on the interior of the esophagus at an esophageal resection site of a second test subject taken after removal of an embodiment of the structural support member device as disclosed herein at 3 to 4 weeks;

FIG. 12B is a photograph of regenerated tissue that is located on the interior of the esophagus at the esophageal resection site of FIG. 12A at an intermediate interval after removal of the structural support member showing tissue growth;

FIG. 12C is a photograph of regenerated tissue that is located on the interior of the esophagus at the esophageal resection site of FIG. 12A at an intermediate interval after removal of the structural support member device showing tissue growth subsequent to the tissue growth depicted in FIG. 12B;

FIG. 12D is a photograph of regenerated tissue that is located on the interior of the esophagus at the esophageal resection site of FIG. 12A at an intermediate interval after removal of the s structural support member device showing tissue growth subsequent to the tissue growth depicted in FIG. 12C;

FIG. 12E is a photograph of regenerated tissue that is located on the interior of the esophagus at the esophageal resection site of FIG. 12A at an intermediate interval after removal of the structural support member device showing tissue growth subsequent to the tissue growth depicted in FIG. 12D;

FIG. 13A is a photograph of tissues from a representative test animal esophagus at 2.5 months post implantation including the surgical site and adjacent distal and proximal tissues excised for histological analysis;

FIG. 13B is a photograph of a magnified cross-sectional sample of mucosa tissue taken from proximal section 1 of FIG. 13A;

FIG. 13C is a photograph of a magnified cross-sectional sample of mucosa tissue taken from proximal section 2 of FIG. 13A;

FIG. 13D is a photograph of a magnified cross-sectional sample of submucosa tissue taken from proximal section 1 of FIG. 13A;

FIG. 13E is a photograph of a magnified cross-sectional sample of submucosa tissue taken from proximal section 2 of FIG. 13A;

FIG. 13F is a photograph of a magnified cross-sectional sample of mucosa tissue taken from distal section 3 of FIG. 13A;

FIG. 13G is a photograph of a magnified cross-sectional sample of mucosa tissue taken from distal section 4 of FIG. 13A;

FIG. 13H a photograph of a magnified cross-sectional sample of mucosa tissue taken from distal section 4 of FIG. 13A;

FIG. 13I a photograph of a magnified cross-sectional sample of submucosa tissue taken from distal section 4 of FIG. 13A;

FIG. 14A is a photograph of tissues of pig esophagus for histological analysis at 2.5 months post implantation with an embodiment of the structural support member as disclosed herein;

FIG. 14B is photograph of a magnified cross-sectional sample taken a section B of FIG. 14A illustrating the presence of mucosal tissue;

FIG. 14C is photograph of a magnified cross-sectional sample taken a section C of FIG. 14A illustrating the presence of mucosal tissue;

FIG. 14D is photograph of a magnified cross-sectional sample taken a section D of FIG. 14A illustrating the presence of mucosal and submucosa tissue and muscular layers;

FIG. 14E is photograph of a magnified cross-sectional sample taken a section E of FIG. 14A illustrating the presence of mucosal and submucosa tissue and muscular layers;

FIG. 14F is a photograph of a cross-sectional sample of esophageal tissue of FIG. 14A used for Ki67 immunoreactivity analysis;

FIG. 14G is a photograph of a cross-sectional sample of esophageal tissue of FIG. 14A used for CD31 immunoreactivity analysis;

FIG. 14H is a photograph of a cross-sectional sample of esophageal tissue of FIG. 14A used for CD3ε immunoreactivity analysis;

FIG. 14I is a photograph of a cross-sectional sample of esophageal tissue of FIG. 14A used for aSMA immunoreactivity analysis;

FIG. 14J is a photograph of a cross-sectional sample of esophageal tissue of FIG. 14A used for Transgelin/SMA22α immunoreactivity analysis;

FIG. 14K is a photograph of a cross-sectional sample of esophageal tissue of FIG. 14A;

FIG. 15A is a photograph of a structural support member and stent at 21 days post implant;

FIG. 15B is an endoscopic image illustrating the lumen of a patent fibrovascular tube of tissue following structural support member and stent removal;

FIG. 16A is a representative gross image of explanted esophagus containing treatment site at necropsy. Esophagus from test animal treated with an embodiment of the synthetic structural support member as disclosed herein at 30-day survival with an arrow indicating the area that is not epithelialized, scale bar equaling 5 cm;

FIG. 16B is a representative gross image of explanted esophagus containing treatment site at necropsy. Esophagus from test animal treated with an embodiment of the synthetic structural support member as disclosed herein at 90-day survival, scale bar equaling 5 cm;

FIG. 16C is a representative gross image of explanted esophagus containing treatment site at necropsy. Esophagus from test animal treated with an embodiment of the synthetic structural support member as disclosed herein at 365-day survival, scale bar equaling 5 cm;

FIG. 16D is a representative gross image of explanted esophagus containing treatment site at necropsy from control animal at 30-day survival with an arrow indicating the area that is not epithelialized, scale bar equaling 5 cm;

FIG. 16E is a representative gross image of explanted esophagus containing treatment site at necropsy from control animal at 30-day survival with an arrow indicating the area that is not epithelialized, scale bar equaling 5 cm;

FIG. 16F is a representative gross image of explanted esophagus containing treatment site at necropsy from control animal at 365-day survival, scale bar equaling 5 cm;

FIG. 17A is a high powered microscopic histological analysis of implants taken a 365-day test recipient take from Zone 2 and visualized using Mason's Trichrome (MT) stain;

FIG. 17B is a high powered microscopic histological analysis of implants taken a 365-day test recipient take from Zone 2 and visualized using SM 2 stain;

FIG. 17C is a high powered microscopic histological analysis of implants taken a 365-day test recipient take from Zone 3 and visualized using MT stain;

FIG. 17D is a high powered microscopic histological analysis of implants taken a 365-day test recipient take from Zone 3D and visualized using SM 2 stain;

FIG. 18A is a micrograph of a cross-section of new tissue growth produced by the method produced in a control and explanted at 30 days post implant respectively stained with Mason's trichrome (MT) stain, cytokeratine-13, CK13; smooth muscle transgelin, SM22; and growth associated protein-43, GAP43 as indicated;

FIG. 18B is a micrograph of a cross-section of new tissue growth produced by the method produced in a test subject implanted with a synthetic structural support member as described in Example III and explanted at 30 days post implant respectively stained with Mason's trichrome (MT) stain, cytokeratine-13, CK13; smooth muscle transgelin, SM22; and growth associated protein-43, GAP43 as indicated;

FIG. 18C is a micrograph of a cross-section of new tissue growth produced by the method produced in a control and explanted at 90 days post implant respectively stained with Mason's trichrome (MT) stain, cytokeratine-13, CK13; smooth muscle transgelin, SM22; and growth associated protein-43, GAP43 as indicated;

FIG. 18D is a micrograph of a cross-section of new tissue growth produced by the method produced in a test subject implanted with a synthetic structural support member as described in Example III and explanted at 90 days post implant respectively stained with Mason's trichrome (MT) stain, cytokeratine-13, CK13; smooth muscle transgelin, SM22; and growth associated protein-43, GAP43 as indicated;

FIG. 18E is a micrograph of a cross-section of new tissue growth produced by the method produced in a control and explanted at 365 days post implant respectively stained with Mason's trichrome (MT) stain, cytokeratine-13, CK13; smooth muscle transgelin, SM22; and growth associated protein-43, GAP43 as indicated;

FIG. 18F is a is a micrograph of a cross-section of new tissue growth produced by the method produced in a test subject implanted with a synthetic structural support member as described in Example III and explanted at 90 days post implant respectively stained with Mason's trichrome (MT) stain, cytokeratine-13, CK13; smooth muscle transgelin, SM22; and growth associated protein-43, GAP43 as indicated; and

FIG. 19 is a process diagram of a second embodiment of the regeneration method as disclosed herein.

### DETAILED DESCRIPTION

Aspects of the disclosure relate in part to the discovery that inserting a synthetic structural support member into the esophageal region of a subject can promote or enhance the regeneration of new esophageal tissue (e.g., a complete and functional esophagus) in the subject without fully incorporating the structural support member into the regenerated tissue. Thus, in some embodiments, the disclosure provides a method for promoting or enhancing growth of gastrointestinal (e.g., esophageal) tissue, the method comprising: delivering to the gastrointestinal (e.g., esophageal) region of a subject a synthetic structural support member, wherein delivery of the synthetic structural support member results in growth of new gastrointestinal (e.g., esophageal) tissue in that region of the subject.

Other aspects of the disclosure relate to the discovery that that inserting a synthetic structural support member into a subject as part of a surgical procedure such as a gastric pull-up operation or the like can reduce or minimize fibrotic scar tissue formation at or near the suture or region of trauma. Such methods and uses can be employed in procedures that include removing a circumferential portion of an esophagus from a subject in which the circumferential portion of the esophagus to be removed is located between the cervical esophagus and the fundus of the stomach forming a cervical-distal anastomosis and a fundal proximal anastomosis, wherein the cervical-distal anastomosis and/or the fundal proximal anastomosis define a native tissue lumenal surface and replacing the removed circumferential portion with a synthetic support structure, having a first end, a second end opposed to the first end and middle section extending therebetween. At least a portion of the synthetic support structure is configured as a tubular member defining an interior lumen and further having an outer polymeric surface extending from the first end to the second end, and a cellularized layer adhering to at least a portion of the outer polymeric surface. The first end of the synthetic support structure is maintained in direct contact and sutured to distal cervical esophageal tissue creating a cervical-synthetic support anastomosis junction. The second end of the synthetic support structure in direct contact and sutured to fundal proximal tissue creating a synthetic support-fundal anastomosis configuration, wherein the first end and the second end are maintained in contact for a period of time sufficient to achieve neo-esophageal tissue growth along the synthetic structural support, the neo-esophageal tissue growth derived from and in contact with the tissue present in the resected organ portion remaining in the subject, the neo-esophageal tissue growth occurring around the synthetic tubular support at a location between the cricophayngeus and the fundal proximal anastomosis of the subject. After neoesphogeal growth is achieved the synthetic structure is removed from contact with the esophagus in a manner such that the neo-esophageal tissue growth is continuous and remains in contact with the cervical-distal region of the esophagus and the fundal proximal portion of the esophagus.

Tissue that is regenerated using methods described herein can be any gastrointestinal tissue, such as tissues of the esophagus, stomach, intestine, colon, rectum, or other hollow gastrointestinal tissue. In some aspects, the disclosure is based, in part, on the surprising discovery that methods described herein result in the regeneration of gastrointestinal tissue comprising muscle tissue, nervous system tissue, or muscle tissue and nervous system tissue.

In some embodiments, the synthetic structural support member is resorbable or dissolvable under physiological conditions (e.g., within a time period corresponding approximately to the time required for tissue regeneration). In some embodiments, at least a portion of the synthetic structural support member is resorbable or dissolvable under suitable physiological conditions.

In some embodiments, the synthetic structural support member is removed from the subject after the formation of a regenerated functional tissue (e.g., esophagus or portion thereof).

In some embodiments, the synthetic structural support member is part of a system that includes a stent or other pressure device configured to be positioned interior of the inserted synthetic structural support member.

In some embodiments, the stent can be positioned contemporaneous with the insertion of the synthetic structural support member. In some embodiments the stent can be removed from the subject after the formation of a regenerated functional tissue (e.g., esophagus or portion thereof). In some embodiments, the stent can be removed contemporaneous with the removal of the synthetic structural support member. In some embodiments, the stent can be removed or repositioned while the synthetic structural support member remains in position. In certain embodiments, the stent can be replaced with an additional stent while the synthetic structural support member remains in position. In certain embodiments, a stent can be placed after the removal of the synthetic structural support member from the subject after the formation of a regenerated functional tissue (e.g., esophagus or portion thereof).

In some embodiments, a structural support member is designed to be readily retrievable by having a) one or more reversible attachments that can be easier to remove than a suture, for example to help disconnect the structural support member from the surrounding tissue (e.g., esophagus) after tissue regeneration, and/or b) one or more features that can be used to help retrieve the structural support member, for example after it has been disconnected from the surrounding tissue (e.g., adjacent esophageal tissue).

Non-limiting examples of reversible attachments include mechanical mechanisms (for example hooks and loops, connectors such as stents, or other mechanical attachments that can be disconnected) and/or chemical mechanisms (for example biodegradable or absorbable attachments and/or attachments that can be selectively removed by chemical or enzymatic means). In some embodiments, absorbable staples can be used. In some embodiments, absorbable staples comprise a co-polymer of polylactide-polyglycolide for example, or any other absorbable blend of material.

In some embodiments, surgical implantation and/or retrieval of a structural support member can be performed with thoracoscopic assistance.

Non-limiting examples of structural features that can assist in the retrieval or removal of a structural support member (e.g., after it is disconnected from the surrounding gastrointestinal tissue) include holes, indents, protrusions, or other structural features, or any combination thereof these structural features are located only on the outer surface of the structural support member. One or more of these structural features can be used to help grip or hold a tool (e.g. a grasper) that is being used to retrieve the structural support member. In some embodiments, one or more of these structural features can be located at only one end of the structural support member (e.g., the end that is proximal to the mouth of the subject). In some embodiments, one or more of these structural features can be located at both ends, or throughout the length of the structural support member. In some embodiments, one or more of these structural features are located only on the outer surface of the structural support member. In some embodiments, one or more of these structural features are located only on the inner surface of the structural support member. In some embodiments, one or more of these structural features are located on both the outer and inner surfaces of the structural support member. In some embodiments, a structural support member is reinforced (e.g., is thicker and/or includes stronger material) at or around the location of one or more structural features that are used to retrieve the structural support member.

In some embodiments, a disconnected structural support member can be removed endoscopically via the lumen of the airway leading to the esophagus. In some embodiments, a disconnected structural support member can be removed surgically.

In some embodiments, the subject has diseased (e.g., cancerous) or injured gastrointestinal tissue that needs to be replaced. In some embodiments, the subject is a human (e.g., a human patient). In some embodiments, the disease may be one of esophagitis, refractory chronic strictures, injury due to caustic burns, perforations, or other injuries can lead to end-stage organ dysfunction requiring surgical repair.

In some embodiments, the subject has congenital anomalies such as congenital anomalies of the esophagus such as esophageal atresia (EA) including but not limited to long gap esophageal atresia (LGEA).

In some embodiments, the disclosure provides engineered structural support members that can be used to replace or repair an esophagus or a portion thereof. In some embodiments, esophageal structural support member s described herein may be used for promoting tissue regeneration (e.g., a regenerated esophagus or portion thereof) to replace a tissue in a subject (e.g., a human). For example, subjects (e.g., a human) having certain cancers (e.g., esophageal cancer) may benefit from replacement of a tissue or organ affected by the cancer. Without wising to be bound by any particular theory, synthetic structural support members described herein promote the growth of new tissue (e.g., esophageal tissue) in a subject and therefore provide a therapeutic benefit to the subject.

In some embodiments, the growth of new esophageal tissue results in the formation of a functional esophagus and or esophagus. Organ junction in the subject. In some embodiments, the new esophageal tissue does not incorporate the structural support member into the regenerated esophageal walls. In some embodiments, the structural support member is designed and manufactured to be absorbable and/or readily retrievable after the esophageal tissue has regenerated. In some embodiments, the structural support member s designed to be at least partially absorbable.

In some embodiments, a synthetic structural support member has a size and shape that approximates the size and shape of a diseased or injured gastrointestinal (e.g., esophageal) region that is being replaced.

In some embodiments, a synthetic structural support member can be employed in corrective procedures in which a portion of the stomach is reformed and joined to the proximal end of the esophagus.

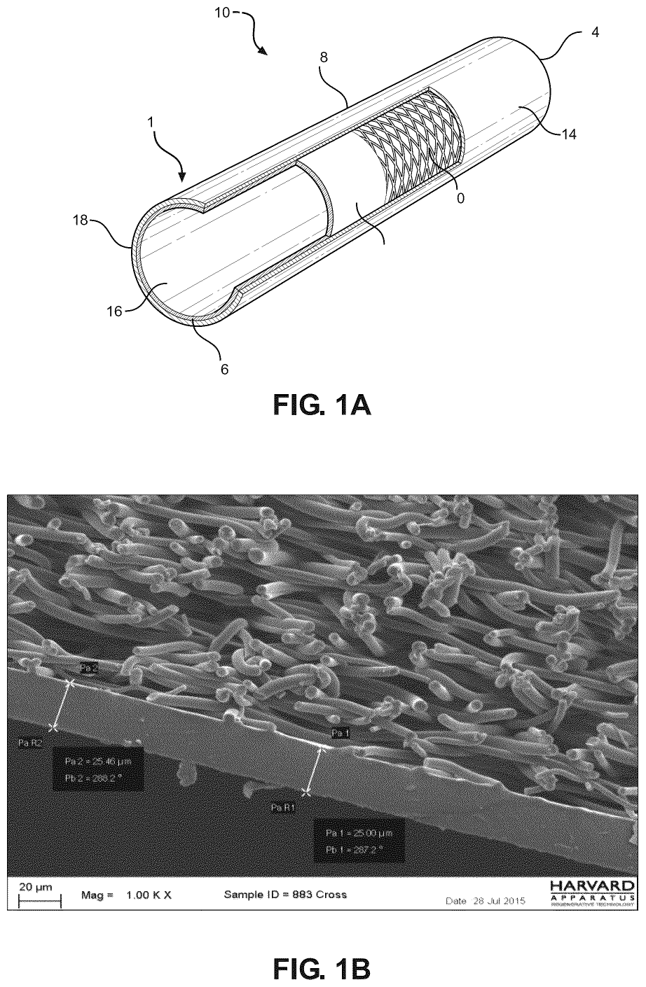

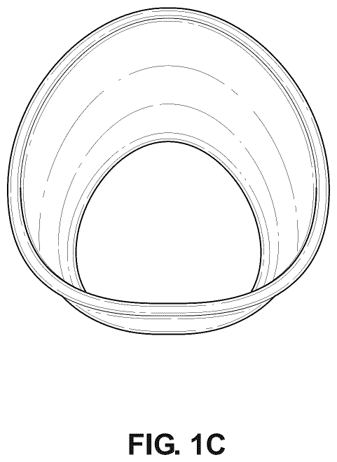

In some embodiments, a structural support member will have at least two layers. The structural support member can have an approximately tubular structure in certain embodiments. FIG. 1A illustrates a non-limiting embodiment of a structural support member 10 having an approximately tubular body 12 having an interiorly oriented surface 14 and an exteriorly oriented surface 16. In some embodiments, a lateral cross-section of the structural support member 10 is approximately circular. In some embodiments, a lateral cross-section is approximately “D” shaped. However, structural support members 10 having other cross-sectional shapes can be used. Structural support member 10 can have any suitable length and diameter depending on the size of the corresponding tissue being regenerated. In some embodiments, a structural support member 10 can be from around 1-10 cms in length (for example 3-6 cms, e.g., about 4 cms) in certain embodiments, or 10-20 cms long in other embodiments. However, it is contemplated that shorter or longer structural support member 10 can be used depending on the application, needs of the patient and/or locations in the gastrointestinal tract requiring treatment. In some embodiments, a structural support member 10 can have an inner diameter of 0.5 to 5 cms. However, structural support members with smaller or larger inner diameters can be used depending on the application, needs of the patient and/or locations in the gastrointestinal tract requiring treatment.

In some embodiments, the length of structural support member 10 can be shorter than the length of a gastrointestinal (e.g., esophageal) region being replaced or replicated. In some embodiments, the structural support member 10 has a length that is 50-95% (for example, about 50-60%, 60-70%, 70-80%, 80-90%, about 80%, about 85%, about 90%, or about 95%) of the length of the tissue being replaced. Without being bound to any theory, it is believed that certain regions of the associated gastrointestinal region can respond positively to traction force exerted on the associated organ tissue resulting the generation of certain bio-organically mediated signals that initiate or promote tissue growth and differentiation.

In certain embodiments, the length of structural support 10 can have a length longer than the length of a gastrointestinal (e.g., esophageal) region being replaced. In some embodiments, the structural support 10 has a length that is between 100% and 150% (for example, about 100-110%, 110-120%, 120-130%, 130-140%, about 100%, about 105%, about 110%, or about 115%) of the length being replaced. It is contemplated that the length of the structural support will be that necessary to effectively replace the effected region. In certain situations, it is contemplated that a structural support 10 will have a length that is longer than the replaced gastrointestinal region to effectively position the structural support and reduce or minimize trauma and ischemia in the effected or associated regions.

In some embodiments, a structural support 10 can be composed of a single layer of synthetic material. However, it is within the purview of this disclosure that the structural support 10 also can include more than one layer of synthetic material.

Accordingly, in some embodiments, the synthetic structural support 10 can be composed of multiple layers (e.g., 2 or more layers, for example 2, 3, 4, 5, or more layers). In some embodiments, one or more layers are made of the same material. In some embodiments, the different layers are made of different materials (e.g., different polymers and/or different polymer arrangements). Synthetic structural supports 10 as disclosed herein may include two or more different components that are assembled to form the structural support as it exists, e.g., prior to cellularization and/or implantation. In some embodiments, a synthetic structural support 10 includes two or more layers that are brought into contact with each other, for example by the synthetic techniques that are used to manufacture the structural support 10. In some embodiments, a structural support 10 may be synthesized using a technique that involves several steps that result in two or more layers being brought together (e.g., the application of a layer of electrospun material onto a portion of the structural support that was previously made, such as an prior layer of electrosprayed material, a prior layer of electrospun material, a surface of a different component (e.g., a braided tube or mesh) that is being incorporated into the structural support, or a combination of two or more thereof).

In the embodiment as depicted in FIG. 1A, structural support 10 includes at least one outer layer 18 that defines the outer surface 14 of the structural support body 12. The structural support 10 includes at least one additional inwardly oriented layer 20. In the embodiment as illustrated, the at least one additional inwardly oriented layer 20 is in direct contact with an inwardly oriented face of the outer layer 18. Where desired or required, the at least one inwardly oriented layer 20 can be configured to provide structural support to the associated structural support body 12. In the embodiment depicted, in FIG. 1 A, the at least one inwardly oriented layer 20 can be configured as a suitable mesh or braid positioned circumferentially around at least a portion of the longitudinal length of the structural support body 12. In other embodiments, it is contemplated that the at least one inwardly oriented layer 20 can be composed of a suitable polymeric layer. In the embodiment as illustrated in FIG. 1A. the body 12 of structural support 10 includes at least one layer 22 that is located interior to the mesh or braid layer 20.

Where desired or required, the structural support 10 can have a wall thickness that is generally uniform. However, in some embodiments, the wall thickness can vary at specific regions of the body 12. In some embodiments, the wall thickness at one or both ends 24, 26 of the body 12 of structural support 10 is different (e.g., thicker) than the walls of the central portion 28 of the structural support 10 (not shown). In some embodiments, the thicker wall regions are stronger and provide greater support for sutures that are connected to one or both ends 24, 26 of the structural support 10 when the structural support is connected to surrounding gastrointestinal tissue. The thicker wall region(s) can also include discrete configurations that facilitate suturing. Non-limiting examples of such configurations include tubes, wholes, etc.

In certain embodiments, at least the exteriorly oriented surface 14 defined on the outwardly oriented layer 18 can be composed of an electrospun polymeric material. In certain embodiments, it is contemplated that the outwardly oriented wall 18 can be composed of electrospun polymeric material. In certain embodiments, the electrospun outwardly oriented layer can be in direct contact with a suitable braid material layer 20.

### Fiber Orientation

Electrospun fibers can be isotropic or anisotropic. In some embodiments, fibers in different layers can have different relative orientations. In some embodiments, fibers in different layers can have substantially the same orientation. Fiber orientation can be altered in each layer of a composite or sandwich structural support in addition.

In some embodiments, structural supports with different porosities can be used. In some embodiments, one or more layers of a structural support permit substantially complete cellular penetration and uniform seeding. In some embodiments, one or more layers of the structural support may be constructed to prevent the penetration of one or more cell types, for example by densely packing the fibers. Controlling fiber diameter can be used to change structural support porosity as the porosity scales with fiber diameter. Alternatively, blends of different polymers may be electrospun together and one polymer preferentially dissolved to increase structural support porosity. The properties of the fibers can be controlled to optimize the fiber diameter, the fiber spacing or porosity, the morphology of each fiber such as the porosity of the fibers or the aspect ratio, varying the shape from round to ribbon-like. In some embodiments, the mechanical properties of each fiber may be controlled or optimized, for example by changing the fiber composition, and/or the degradation rate.

In certain embodiments, the electrospun fiber material can provide a contoured surface such as a that depicted in FIG. 1B. In certain embodiments, at least one electrospun layer in structural support 10 can be a polymeric fiber material such as polycarbonate polyurethane and can be produced by dissolving polycarbonate-polyurethane in a suitable solvent such as hexa fluoroisopropanol (HFIP) that is spun and dried.

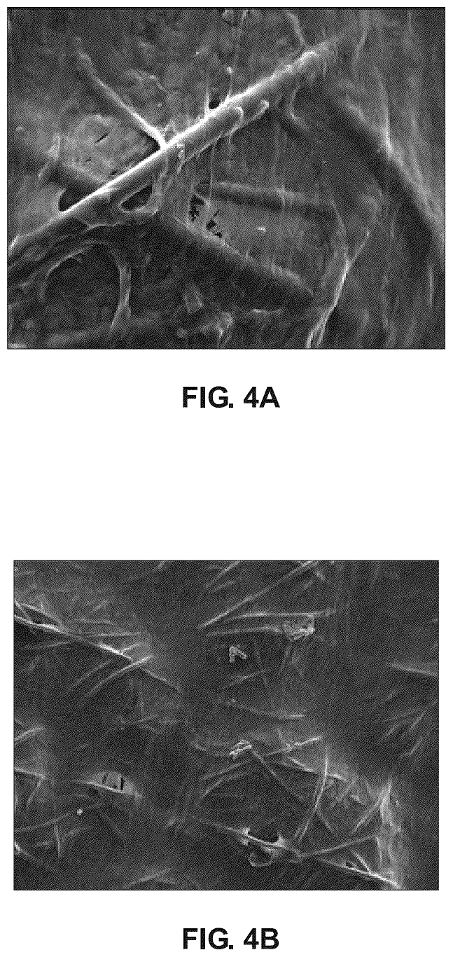

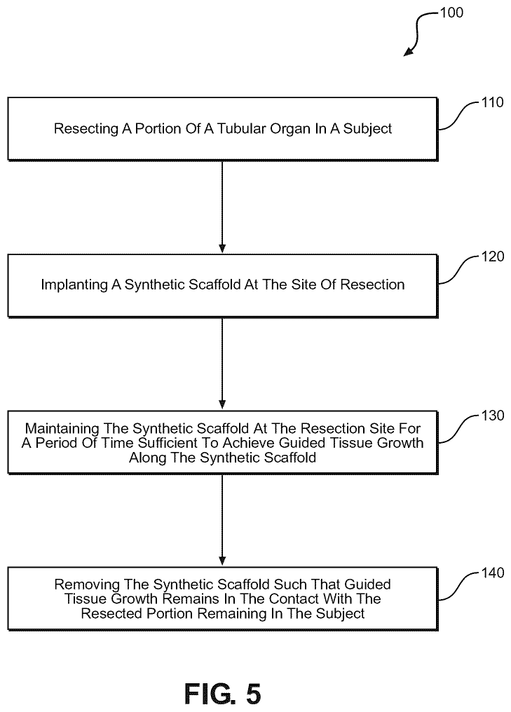

The spacing and porosity of the electrospun fiber material can be that such that cells seeded on the structural support surface can adhere in suspended overlying relationship between respective fibers to permit the seeded cellular material to form sheets thereon as illustrated in FIGS. 4A and 4B.

### Layering of Synthetic Structural Supports

Aspects of the disclosure relate to methods for producing synthetic structural supports. In some embodiments, tubular synthetic structural supports (e.g., a synthetic esophageal structural support) are produced on a mandrel (e.g., by depositing material via electrospraying and/or electrospinning).

In some embodiments, one or more layers of a synthetic structural support provide structural support to the structural support, conferring a desired mechanical property to the structural support. In some embodiments, a braided material (e.g., a braided tube, for example a nitinol braid, a PET braid, or a braid of other metallic or non-metallic material) can be inserted between two different layers of a structural support to provide structural support. The compression force of the braided material (e.g., the force that the braid can exert on the next layer of material, for example the outer electrospun layer of material) can be controlled by controlling the pick count of the braid. In some embodiments, a braid can be coated (e.g., by dipping or other technique) in an organic solvent to help attach it to one or more other layers of the structural support 10. In some embodiments, the length of the braid 20 does not extend to the ends of the structural support body 12. In some embodiments, one or both ends of the structural support 10 consist of two or more layers of material without a braided layer, whereas the central portion 28 of the structural support body 12 includes an additional braided layer.

In some embodiments, one or more layers of a synthetic structural support provide a barrier in the structural support, creating a separation (e.g., a relatively impermeable separation) between an inner space (e.g., a lumenal space) and an external space. In some embodiments, a barrier can be an electrosprayed polyurethane (PU) layer.

In some embodiments, different layers of a structural support 10 can include one or more polymers (e.g., polyethylene terephthalate (PET), PU, or blends of PET and PU). In some embodiments, a structural support 10 can include a nitinol braid sandwiched between an inner PU layer (e.g., that was electrosprayed or electrospun onto a mandrel) and an outer PU layer (e.g., that was electrosprayed onto the braided material).

In certain embodiments the structural support 10 can be formed using a structural support or mandrel. In some embodiments, a structural support or mandrel may be coated with a material (e.g., PLGA or other polymer) prior to depositing one or more layers of PU, PET, or a combination thereof.

In certain embodiments, the material in the braid or mesh layer can be composed of absorbable polymeric material.

### Structural Support Production-Fiber Materials

In some embodiments, one or more layers of a structural support may be constructed from fibrous material. In some embodiments, structural supports comprise one or more types of fiber (e.g., nanofibers). In some embodiments, structural supports comprise one or more natural fibers, one or more synthetic fibers, one or more polymers, or any combination thereof. It should be appreciated that different material (e.g., different fibers) can be used in methods and compositions described herein. In some embodiments, the material is biocompatible so that it can support cell growth. In some embodiments, the material is permanent, semi-permanent (e.g., it persists for several years after implantation into the host), or rapidly degradable (e.g., it is resorbed within several weeks or months after implantation into the host).

In some embodiments, the structural support comprises or consists of electrospun material (e.g., macro or nanofibers). In some embodiments, the electrospun material contains or consists of PET (polyethylene terephthalate (sometimes written poly(ethylene terephthalate)). In some embodiments, the electrospun material contains or consists of polyurethane (PU). In some embodiments, the electrospun material contains or consists of PET and PU.

In some embodiments, the artificial structural support may consist of or include one or more of any of the following materials: elastic polymers (e.g., one or more polyurethanes (PU), for example polycarbonates and/or polyesters), acrylamide polymers, Nylon, resorbable polysulfone polymers and mixtures thereof. In some embodiments, the structural support may consist of or include polyethylene, polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinylalcohol in various degrees of hydrolysis (e.g., 87% to 99.5%) in cross-linked and non-cross-linked forms. In certain embodiments, the polymeric compound can also include compounds or processes to increase the hydrophilic nature of the polymer. In certain embodiments, this can involve incorporating compounds such as block copolymers based on ethylene oxide and propylene oxide. It is also contemplated that the hydrophilic nature of the polymer can be increase by suitable plasma treatment if desired or required.

In some embodiments, the structural support may consist of or include block copolymers. In some embodiments, addition polymers like polyvinylidene fluoride, syndiotactic polystyrene, copolymer of vinylidene fluoride and hexafluoropropylene, polyvinyl alcohol, polyvinyl acetate, amorphous addition polymers, such as poly(acrylonitrile) and its copolymers with acrylic acid and methacrylate, polystyrene, poly(vinyl chloride) and its various copolymers, poly(methyl methacrylate) and its various copolymers, and PET (polyethylene terephthalate (sometimes written poly(ethylene terephthalate)) can be solution spun or electrospun and combined with any other material disclosed herein to produce a structural support. In some embodiments, highly crystalline polymers like polyethylene and polypropylene may be solution spun or combined with any other material disclosed herein to produce a structural support.

In some embodiments, one or more polymers are modified to reduce their hydrophobicity and/or increase their hydrophilicity after the structural support synthesis, but before structural support cellularization and/or implantation.

The electrospun fibers can have a dimeter less than 10 micrometers in certain embodiments. In certain embodiments, the electrospun fibers. In certain embodiments, the electrospun fibers can have a diameter between 3 and 10 micrometers. The electrospun fibers can have a dimeter between 3 and 5 micrometers in certain embodiments.

In certain embodiments, it is contemplated that the material in the braid layer can be made in whole or in part of bioabsorbable materials such as PLGA and the like. it is also contemplated that, in certain configurations, the braid material can be loaded materials and compounds that can promote and/or support tissue growth and regeneration. Non-limiting examples of such compounds and materials include one or more of the following: antibiotics, growth factors and the like.

### Electrospinning

In some embodiments, structural supports are produced that include one or more layers (e.g., of PU and/or PET) produced via electrospinning. Electrospun material can be used for a variety of applications, including as a structural support for tissue engineering. Appropriate methods of electrospinning polymers may include those described in Doshi and Reneker. Electrospinning process and application of electrospun fibers. J Electrostat. 1995; 35:151-60.; Reneker D H, Chun I. Nanometer diameter fibers of polymer produced by electrospinning. Nanotechnology. 1996; 7:216-23; Dzenis Y. Spinning continuous fibers for nanotechnology. Science. 2004; 304:1917-19; or Vasita and Katti. Nanofibers and their applications in tissue engineering. Int J. Nanomedicine. 2006; 1(1): 15-30, the contents of which relating to electrospinning are incorporated herein by reference. Electrospinning is a versatile technique that can be used to produce either randomly oriented or aligned fibers with essentially any chemistry and diameters ranging from nm scale (e.g., around 15 nm) to micron scale (e.g., around 10 microns).

In some embodiments, electrospinning and electrospraying techniques used herein involve using a high voltage electric field to charge a polymer solution (or melt) that is delivered through a nozzle (e.g., as a jet of polymer solution) and deposited on a target surface. The target surface can be the surface of a static plate, a rotating drum (e.g., mandrel), or other form of collector surface that is both electrically conductive and electrically grounded so that the charged polymer solution is drawn towards the surface.

In some embodiments, the electric field employed is typically on the order of several kV, and the distance between the nozzle and the target surface is usually several cm or more. The solvent of the polymer solution evaporates (at least partially) between leaving the nozzle and reaching the target surface. This results in the deposition of polymer fibers on the surface. Typical fiber diameters range from several nanometers to several microns. The relative orientation of the fibers can be affected by the movement of the target surface relative to the nozzle. For example, if the target surface is the surface of a rotating mandrel, the fibers will align (at least partially) on the surface in the direction of rotation. In some cases, the nozzle can be scanned back and forth between both ends of a rotating mandrel.

In some embodiments, the size and density of the polymer fibers, the extent of fiber alignment, and other physical characteristics of an electrospun material can be impacted by factors including, but not limited to, the nature of the polymer solution, the size of the nozzle, the electrical field, the distance between the nozzle and the target surface, the properties of the target surface, the relative movement (e.g., distance and/or speed) between the nozzle and the target surface, and other factors that can affect solvent evaporation and polymer deposition.

Electrospinning and electrospraying processes may be used for producing interlinked polymer fiber structural supports (e.g., hollow synthetic structural supports) on a mandrel.

### Support/Mandrel

In some embodiments, structural support 10 (e.g., a structural support having two or more layers) can be produced using a support (e.g., a solid or hollow support) on which the structural support 10 can be formed. For example, a support can be an electrospinning collector, for example a mandrel, or a tube, or any other shaped support. It should be appreciated that the support can have any size or shape. However, in some embodiments, the size and shape of the support is designed to produce a structural support that will support an artificial tissue of the same or similar size as the gastrointestinal tissue (or portion thereof) being replaced or supplemented in a host. It should be appreciated that a mandrel for electrospinning should have a conductive surface. In some embodiments, an electrospinning mandrel is made of a conductive material (e.g., including one or more metals). However, in some embodiments, an electrospinning mandrel includes a conductive coating (e.g., including one or more metals) covering a non-conductive central support.

It has been found quite unexpectedly that positioning suitable braid material to be integrated in the resulting structural support 10 at a location proximate to the surface of the mandrel can serve as an aid to facilitate removal of the resulting structural support 10 from contact with the mandrel.

### Structural Support Properties

It should be appreciated that aspects of the disclosure are useful for enhancing the physical and functional properties of any structural support, for example a structural support based on electrospun and/or electro sprayed fibers. In some embodiments, one or more structural support components can be thin sheets, cylinders, thick ribs, solid blocks, branched networks, etc., or any combination thereof having different dimensions. In some embodiments, the dimensions of a complete and/or assembled structural support are similar or identical to the dimension of a tissue or organ being replaced. In some embodiments, individual components or layers of a structural support have smaller dimensions. For example, the thickness of a nanofiber layer can be from several nm to 100 nm, to 1-1000 microns, or even several mm. However, in some embodiments, the dimensions of one or more structural support components can be from about 1 mm to 50 cms. However, larger, smaller, or intermediate sized structures may be made as described herein.

In some embodiments, structural supports are formed as tubular structures that can be seeded with cells to form tubular tissue regions (e.g., esophageal, or other tubular regions). It should be appreciated that a tubular region can be a cylinder with a uniform diameter. However, in some embodiments, a tubular region can have any appropriate tubular shape (for example, including portions with different diameters along the length of the tubular region). A tubular region also can include a branch or a series of branches. In some embodiments, a tubular structural support is produced having an opening at one end, both ends, or a plurality of ends (e.g., in the case of a branched structural support). However, a tubular structural support may be closed at one, both, or all ends, as aspects of the invention are not limited in this respect. It also should be appreciated that aspects of the invention may be used to produce structural supports for any type or organ, including hollow and solid organs, as the invention is not limited in this respect. In some embodiments, aspects of the invention are useful to enhance the stability of structural support or other structures that include two or more regions or layers of fibers (e.g., electrospun nanofibers) that are not physically connected.

In some embodiments, a structural support is designed to have a porous surface having pores ranging from around 10 nm to about 100 microns in diameter that can promote cellularization. In some embodiments, pores have an average pore diameter of less than 50 microns, less than 40 microns, less than 30 microns, less than 20 microns or less than 10 microns (e.g., approximately 5, approximately 10, or approximately 15 microns). In some embodiments, pores have an average pore diameter of 20-40 microns. In some embodiments, pore size is selected to prevent or reduce an immune response or other unwanted host response in the subject. Pore sizes can be estimated using computational and/or experimental techniques (e.g., using porosimetry). However, it should be appreciated that pores of other sizes also can be included.

In some embodiments, a surface layer of a structural support is synthesized using fibers that include one or more dissolvable particles that can be dissolved during or after synthesis (e.g., by exposure to a solvent, an aqueous solution, for example, water or a buffer) to leave behind pores the size of the dissolvable particles. In some embodiments, the particles are included in the polymer mix that is pumped to the nozzle of an electrospinning device. As a result, the particles are deposited along with the fibers. In some embodiments, the electrospinning procedure is configured to deposit thick fibers (e.g., having an average fiber diameter of several microns, about 10 microns, and thicker). In some embodiments, if the fibers are deposited in a dense pattern, one or more fibers will merge prior to curing to form larger macrostructures (e.g., 10-100 microns thick or more). In some embodiments, these macrostructures can entangle two or more layers of fibers and or portions (e.g., fibers) from two or more different components of a structural support thereby increasing the mechanical integrity of the structural support. In some embodiments, when such macrostructures are formed (e.g., via electrospinning as described herein) at one or more stages during structural support synthesis (e.g., to connect two or more layers and/or components), the surface of the macrostructure(s) can be treated (e.g., etched or made porous using dissolvable particles as described herein) in order to provide a surface suitable for cellularization.

In some embodiments, the amount of flexible structural support material (e.g., the slack) between two or more structural components (e.g., rings), between structural members (e.g., arcuate members) of a single continuous structural component, and/or of a braided support material can be used to determine the mechanical properties (e.g., tensile strength, elongation, rotation, compression, range of motion, bending, resistance, compliance, degrees of freedom, elasticity, or any other mechanical property, or a combination thereof) of a synthetic structural support.

In certain embodiments, the structural support 10 can also include a cellular sheath derived from cells seeded on the outer surface of the structural support during incubation. The cellular sheath adheres to and is in overlying relationship to the outer surface of the structural support. It is contemplated that a major portion of the cells present in the cellular sheath will be connected to the outermost surface of the outer surface and will span pores defined therein to form a continuous or generally continuous surface.

In certain embodiments, the cellular sheath can have a thickness sufficient to provide structural integrity to the sheath layer. In certain embodiments, the cellular sheath will be composed of a number of cells which are in contact with the external surface of the structural support sufficient to direct regenerating cells in contact with the sheath to produce a tissue wall that overlays the sheath but does not integrate therewith. In certain embodiments, the sheath can be composed of a lining that is between 1 and 100 cells thick on average. Certain embodiments can have a cell thickness between 10 and 100; between 10 and 30; between 20 and 30, between 20 and 40; between 20 and 50; between 10 and 20; between 30 and 50; between 30 and 60; between 40 and 60; between 40 and 70; between 70 and 90.

The structural support 10 with the associated cellular sheath provides a moveable insertable device that can be positioned in a suitable gastrointestinal resection site. The structural support 10 with the associated cellular sheath in contact therewith can be transported to the desired resection site for implantation. In certain embodiments, the structural support 10 is configured to be removable from the implantation site after suitable regeneration of the resected organ. In certain embodiments, the removed structural support will include some or all of the cellular sheath connected thereto.

Also disclosed are various embodiments of method of regenerating a tubular organ such as a gastrointestinal organ. In certain embodiments, the method 100 includes the step of resecting that comprises resecting a portion of a tubular organ in a subject as at reference numeral 110. The organ to be resected can be a tubular organ of the gastrointestinal tract that has been damaged or compromised by disease injury, trauma or congenital conditions. In certain embodiments, non-limiting examples of suitable organs include one of the esophagus, rectum and the like. In certain embodiments, suitable organs include at least one of the esophagus, small intestines, colon, rectum.

The resection can be achieved by any suitable surgical procedure and produced a resected organ portion that remains connected to the gastrointestinal tract and remains in the subject after resection. The resection operation can yield suitable resection edges in certain embodiments.

After resection is completed, a synthetic structural support is implanted at the site of the resection as at reference numeral 120. In certain embodiments, implantation can include the step of connecting the respective ends of the resected organ as it remains in the subject to respective ends of the synthetic structural support such that the synthetic structural support and at the resected organ can achieve a suitable junction between the respective members. This can be achieved by one or more of sutures, bioorganic tissue glue, etc.

In certain embodiments, the synthetic structural support that is implanted can be a tubular member that has an outer polymeric surface and a cellularized sheath layer overlying at least a portion of the of the outer polymeric surface. Various embodiments of the synthetic structural support have been discussed and can be employed and utilized in the method disclosed herein. In certain embodiments, the synthetic structural support will include a first end and a second end opposed to the first end, an outer polymeric surface positioned between the first end and the second end and a cellularized sheath layer overlying at least a portion of the outer polymeric surface. In certain embodiments, the implantation step can be one that brings at least a portion of the cellularized sheath layer into proximate contact with to at least one of the resection edges of the resected organ portion.

In certain embodiments, the method as disclosed herein also includes the step of maintaining the synthetic structural support at the resection site for a period of time sufficient to achieve guided tissue growth along the synthetic structural support as at reference numeral 130. In certain embodiments, the guided tissue growth is derived from and is in contact with the tissue present in the resected organ portion remaining in the subject. In certain embodiments, the guided tissue growth will be contiguous with the associated regions of the resected organ. In certain embodiments, the guided tissue growth will exhibit differentiated tissue. In certain embodiments, the guided tissue growth will parallel the outer surface of the cellularized sheath layer at a position outward thereof. In certain embodiments, the guided tissue growth is derived from and is in contact with the tissue present in the resected organ portion remaining in the subject and will be contiguous with the associated regions of the resected organ. The guided tissue growth will exhibit differentiated tissue growth and can be parallel the outer surface of the cellularized sheath layer at a position outward thereof.

After the guided tissue growth has been achieved, the process 100 as disclosed herein can include step of removing the synthetic structural support as at reference numeral 140. In certain embodiments, the removing step occurs in a manner such that the guided tissue growth remains in the contact with the resected portion of the organ remaining in the subject. In certain embodiments, the removal process can include intrascopically removing the synthetic structural support from the interior of the guided tissue growth as well as various endoscopic procedures.

In certain embodiments, the synthetic structural support can be constructed in whole or in part from bioabsorbable polymeric material. In such situations, the method as disclosed herein can include the step of maintaining contact between the synthetic structural support and the resection edge for an intervals sufficient to achieve guided tissue growth along the synthetic structural support such that at least a portion of the synthetic structural support is absorbed at the site of resection within a period of time sufficient to achieve guided tissue growth along the synthetic structural support. In certain embodiments where the structural support is composed entirely of bioabsorbable material, the structural support will be configured to maintain structural integrity during guided tissue growth. In certain embodiments, where the synthetic structural support is composed of bioabsorbable material in selected regions, it is contemplated that the remainder of the structural support can be removed by suitable procedures after the guided tissue growth has been achieved.

Guided tissue growth can be monitored by suitable means. In certain embodiments, tissue growth can be monitored endoscopically.

In certain embodiments, the method 200 includes the step of joining a portion of a first tubular organ present in a system to a second tubular organ present in the system. The method 200 includes the step of preparing the first tubular organ as at reference numeral 210 and the step of preparing the second tubular organ as at reference number 220. The first and second tubular organs can be those present in a system such as the gastrointestinal tract of a subject. In certain embodiments, suitable organs include at least two organs sequentially present in the system such as the esophagus, stomach, small intestines, colon, rectum. In certain embodiments, the organs to be joined can be those sequentially positioned in the gastrointestinal tracts such as esophagus to stomach, stomach to small intestine, small intestine to colon, etc.

At least one of the two organs to be prepared can be a tubular organ of the gastrointestinal tract that has been damaged or compromised by disease injury, trauma or congenital conditions. In certain embodiments, the damaged tubular organ can be the esophagus. One or both preparation steps 210, 220 may include resection of a portion of the respective tubular organ. One or both preparation steps 210, 220 may include formation of a junction portion one or both of the associated tubular organs. Formation processes can be any that would be known to the skilled artisan. Non-limiting examples include stretching, modeling and the like to produce anastomosis sites.

When the preparation steps 210, 220 are complete, the respective anastomoses can be connected either directly to one another or to the synthetic structural support 10 as disclosed herein as at reference numeral 230. In certain embodiments where the respective ends of the first and second tubular organ can be directly connected as at the respective anastomoses and the synthesis structural support 10 may be positioned in place subsequent to the connection of the respective ends such that the synthetic structural support 10 underlies the junction that has been formed. In certain embodiments, the synthetic structural support 10 can be inserted endoscopically and anchored by suitable means. In certain embodiments, a stent such as stent 50 can be inserted into position after the synthetic structural support 10 is in position. Insertion of the stent 50 can be accompanied by other anchoring steps such as suturing or the like. It is also within the purview of this disclosure that stent insertion can be independent of other anchoring methods.

In certain embodiments of the method as disclosed herein, the method can also include the step of imparting cellular material onto the polymeric surface of the synthetic structural support and allowing the cellular material to grow to form a cellularized material layer such as a cellular sheath layer, the imparting and allowing steps occurring prior to the resecting step.

In certain embodiments, the synthetic structural support that is employed in the method disclosed herein is a tubular member where the outer surface includes spun polymeric fibers. In certain embodiments, the spun fibers can be electrospun by suitable methods such as those described in this disclosure. The cellularized sheath layer spans at least a portion outwardly positioned electrospun fibers in certain embodiments. The cellularized sheath layer can is composed of cellular material, the cellular material including at least one of mesenchymal cells, stem cells, pluripotent cells. The cellular material can be autologously derived from the subject or can be allogenically derived.

Without being bound to any theory, it is believed that implanting a synthetic structural support such as those variously disclosed herein, particularly one seeded with an overlying cellular sheath, promotes growth, regeneration and differentiation of the subject tissue in contact with or proximate to the location of the implanted synthetic structural support. The growing regenerating tissue is guided by the synthetic structural support and associated cellular sheath to produce a tubular cellular body that is integrally connected to the resected ends of the remaining tubular organ(s) and outwardly flaring to encapsulate the synthetic structural support and associated cellular sheath layer. It is believed that the structural support and associated cellular sheath layer may promote or stimulate regenerative growth of the resected tissue while minimizing tissue rejection responses. It is also believed that the presence of the cellular sheath layer can reduce or minimize penetration of the regenerated tissue into the sheath layer during growth and differentiation. In certain embodiments, tissue generation proceeds from the respective ends toward the middle.

Without being bound to any theory, it is believed that, once implanted, the deposition of cellular material on the surface of the synthetic support structure may provide signaling into the native tissue proximate to the anastomosis such as the associated cellular niche for organized regrowth of differentiated tissue structure. It is also believed that cellular material present in the cellularized sheath layer can facilitate tissue remodeling that can reduce the incidence of fibrotic scar tissue present at or proximate to the surgical site after removal of the implanted synthetic cellular support. In certain embodiments, the remodeling and/or reduction in fibrotic scar tissue can be evidenced during the presence of the implanted synthetic support structure. In certain embodiments, the remodeling and/or reduction in initial fibrotic scar tissue can continue to proceed after removal of the implanted synthetic support. In certain embodiments, the remodeling and/or reduction in fibrotic scar tissue can be evidenced at 90 days post-surgery; 120 days post-surgery, 365 days post-surgery. In certain embodiments, the reduction in fibrotic scar tissue formation and/or continued induced remodeling of fibroid scar tissue initiated by the presence of the synthetic support structure can contribute to the reduction and/or elimination of strictures at or near the surgical site.

In certain embodiments, the synthetic support structure may include other compounds that can stimulate the deposition of cells/tissue from the recipient circumferentially and longitudinally around and along the entire synthetic structure. Non-limiting examples of such compounds may include add VEGF, MMP2 and IL-8.

Once the regenerated tissue is in position, the synthetic structural support can be removed. In certain embodiments, immediately after the removal of the synthetic structural support, the regenerated tissue structure will lack the inner epithelial layer. This layer has been seen to regenerate after removal of the structural support as illustrated in FIGS. 11A, 11B and 11C taken immediately after structural support removal, 2 months post removal and 3 months post removal respectively.

The synthetic support structures disclosed herein can be advantageously employed in surgical procedures in gastrointestinal tract of a subject such as those occurring between the cricopharyngeal notch and a suprasternal notch. The synthetic support structure as disclosed herein can have a first end, a second end opposed to the first end and middle section extending between the first end and the second end. The synthetic support structure is configured as a tubular member defining an interior lumen. the synthetic support structure further having an outer polymeric surface extending from the first end to the second end, and a cellularized layer adhering to at least a portion of the outer polymeric surface.

The procedures can include removal of a portion of the esophagus from the subject, particularly removal of a portion of the esophagus located between the cervical esophagus region and the fundus of the stomach to from a cervical-distal anastomosis and a fundal proximal anastomosis such that the cervical-distal anastomosis and/or the fundal proximal anastomosis define a native tissue lumenal surface. Where desired or required, the procedure can involve removal of a circumferential portion of the esophagus in the defined region to form the cervical-distal anastomosis and the fundal proximal anastomosis.

In the surgical procedure, the portion of the synthetic support structure as disclosed herein replaces the removed portion of the esophagus and the first end of the synthetic support structure is maintained in direct contact and connected to the distal cervical esophageal tissue that creates a junction such as a cervical tissue-synthetic support anastomosis junction. The second end of the synthetic support structure is maintained in direct contact and connected to fundal proximal tissue thereby creating a synthetic support-fundal anastomosis. Where desired or required, one or both of the connections can be accomplished by sutures.

The first end of the synthetic support structure and the second end of the support structure. The first and second end of the support structure are maintained in contact with the associated tissue for a period of time sufficient to achieve neo-esophageal tissue growth along the synthetic structural support. “Contact” as that term is employed can be direct end-to-end connection between one or both ends or can include positioned in the respective ends proximate to the associated anastomosis in certain applications. The neo-esophageal tissue growth accomplished is derived from and in contact with the tissue present in the resected organ portion remaining in the subject and can occur around the synthetic tubular support at a location associated with a suitable stem cell niche such as between the cricophayngeus and the fundal proximal anastomosis of the subject.

After neoesophageal tissue growth has been achieved, the synthetic support structure can be removed from contact with the esophagus in a manner such that the neo-esophageal tissue growth is continuous and remains in contact with the cervical-distal portion of the esophagus and the fundal proximal portion of the esophagus. In certain embodiments, the synthetic support structure can be removed intrascopically as by using endoscopic techniques. Neoesophageal tissue growth can be guided tissue growth that epithelial tissue, smooth muscle tissue vascular tissue and neuronal cellular proteins. In certain embodiments the guided tissue growth including epithelial tissue, smooth muscle tissue vascular tissue and neuronal cellular proteins overlies the outer surface of the tubular synthetic support structure and may over lie in a manner that does not adhere to the outer polymeric surface of the tubular synthetic support structure.

Where desired or required, the synthetic support structure can be part of an assembly that includes at least one pressure member and the method can include the step of positioning the at least one pressure member the lumen of the synthetic support structure after surgical placement of the synthetic support structure. Non-limiting examples of a suitable pressure member include stents, nasogastric tubes and the like. The pressure member can be configured such that the ends of the pressure member extends beyond the one or more of the anastomosis sites such that the support structure is interposed between the pressure member and the luminal surface of native tissue after surgical placement of the synthetic support structure. In other embodiments, the first pressure member spans at least one junction between at least one anastomosis and at least one end of the synthetic support structure when in position. In certain embodiments, the first pressure member spans at least one junction between the respective anastomoses and both ends of the synthetic support structure when in position. In certain embodiments, the first pressure member spans a junction between the cervical-distal anastomosis and the first end of the tubular synthetic support structure and a junction between the fundal proximal anastomosis and the second end of the tubular synthetic support structure

In certain procedures the synthesis support structure assembly can include a second pressure member in addition to the first pressure member discussed. In certain procedures, the first pressure member and the synthetic support structure can be removed after guided tissue growth has been achieved, in particular between the cricophayngeus notch and the suprasternal notch or other stem cell niches. The second pressure member can be positioned in the desired location subsequent to the removal of the first pressure member and can remain in position for an interval sufficient to permit epithelialization of the lumenal surface of the neo-tissue. Where desired or required, the second pressure member can be configured as a stent or a nasogastric tube. In certain embodiments, the step of positioning a second pressure member in the esophagus at the region defined by guided tissue grow at a time subsequent to the step of removing the first pressure member, the second pressure member remaining in position for an interval of at least 15 days.

In certain embodiments, the process as outlined can involve the step of translocating the fundal proximal region of the stomach of the subject to a position above the diaphragm of the subject. Examples of such process include those associated with stomach pull-up procedures.

In order to further understand the present disclosure, reference is made to the following Examples. These Examples are included for purposes of illustration and are to be considered illustrative of the present disclosure and the invention as set forth in the claims.

### EXAMPLES

### Example 1: Esophageal Structural Supports

Synthetic esophageal structural supports were produced containing three layers of material as illustrated in FIG. 1A. A first layer of polyurethane (PU) was deposited onto a metallic mandrel via electrospraying. A braided material was then deposited on the first PU layer. A second PU layer was then deposited via electrospinning. The resulting structural supports were then removed from the mandrel. Each structural support defined a tubular structure having a wall that included three layers (a braided layer sandwiched between and inner electrosprayed layer and an outer electrospun layer). Physical dimensions of the structural support were determined by scanning electron microscopy (SEM). The average structural support wall thickness was approximately 500 microns. A non-limiting SEM view of a cross-section of the wall is shown in FIG. 1B. A non-limiting visual image of a cross-section of the tubular structural support is shown in FIG. 1C. This image shows that the cross-section is approximately “D” shaped. This can be obtained by using a mandrel that has a “D” shaped cross section.

The outer electrospun layer was a layer of polymer fibers defining pores. The average fiber diameter in the outer layer was approximately 3-6 microns The average pore size was approximately 15-20 microns, and the median pore size was approximately 25-45 microns.

Structural supports were attached to a support capable of rotating in a bath of liquid medium within a bioreactor chamber. The rotating mechanism can include magnetic drives that allow the support along with the attached structural support to be rotated around its longitudinal axis within the liquid bath.

Structural supports were seeded with cells (e.g., MSCs or other stem cells) by depositing cell solutions on the external structural support surface. The seeded structural supports were then incubated in liquid media that supports cell growth by rotating the structural supports in a bath of the liquid media within a bioreactor chamber for approximately one week. The resulting structural supports include a cellular sheath that is in overlying relationship to the outer surface of the structural support. In certain embodiments, the cellular sheath can have a thickness sufficient to provide structural integrity to the sheath layer. In certain embodiments, the cellular sheath will be composed of a number of cells which are in contact with the external surface of the structural support sufficient to direct regenerating cells in contact with the sheath to produce a tissue wall that overlays the sheath but does not integrate therewith. In certain embodiments, the sheath can be composed of a lining that is between 1 and 100 cells thick on average. Certain embodiments can have a cell thickness between 10 and 100; between 10 and 30; between 20 and 30, between 20 and 40; between 20 and 50; between 10 and 20; between 30 and 50; between 30 and 60; between 40 and 60; between 40 and 70; between 70 and 90.

The structural support 10 having the seeded cellular sheath can be implanted into the resection site and can be positioned in place. It is contemplated that one or more of the seeded cell populations present in the cellular sheath can continued to grow post implantation. In such situations, the seeded cells present in the cellular sheath will maintain and support a structure that is separate from and tandem to the tissue regenerating at the implantation site.

In the present example, the respective structural supports with associated cellular sheaths were then implanted into esophageal sites in pigs. An approximately 5 cm section of esophagus was removed and replaced with a structural support section that was sutured to the ends of the remaining esophageal tissue in the subject.

The regeneration of esophageal tissue was monitored endoscopically for several weeks.

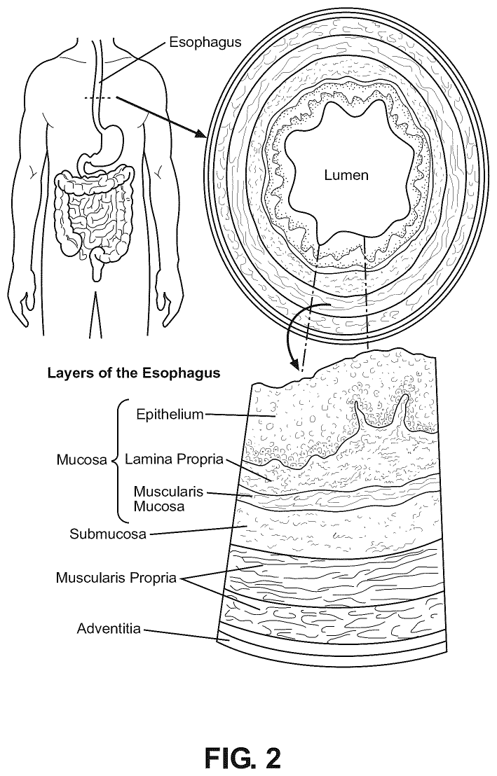

The esophagus is a long muscular tube that has cervical, thoracic, and abdominal regions. FIG. 2 is a diagram that illustrates a cross-section of an esophagus in a human. In an adult human the esophagus can be 18 cm to 25 cm in length. An esophagus wall is composed of striated muscle in the upper part, smooth muscle in the lower part, and a mixture of the two in the middle. Accordingly, provided herein, in some embodiments, are multilayered synthetic structural supports that can promote repair and regeneration of esophageal tissue having two or more layers corresponding to natural esophageal tissue layers.

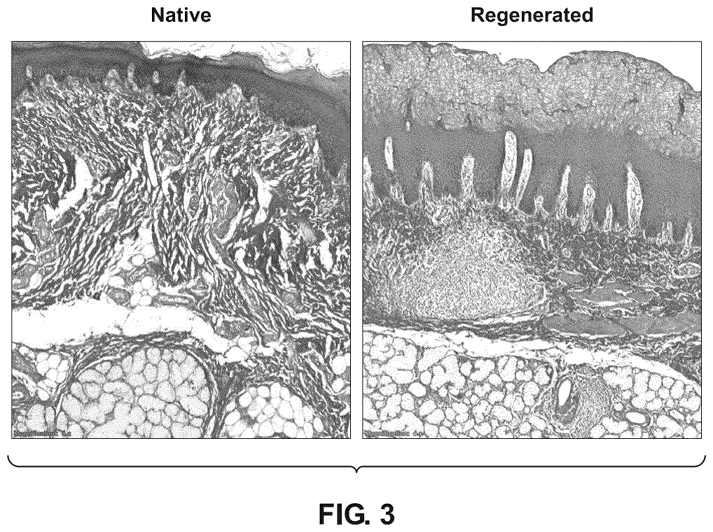

FIG. 3 shows stained cross-sections of native and regenerated esophageal tissue 1-2 weeks after an esophageal structural support implant in a pig. The cross section shows regeneration of essentially all the esophageal tissue layers (including different muscle and gland layers). Further analysis of the regenerated tissue revealed that the structural support itself was not incorporated into the regenerated esophageal wall. The structural support was still present within the esophagus but appeared to have acted as a guide that stimulated esophageal regeneration as opposed to becoming an integral part of the regenerated esophagus.

### Example II. Esophageal Implant

Synthetic esophageal structural supports were produced that contained three layers as illustrated in FIG. 1A with the outer electrospun layer of poly-carbonate-polyurethane being deposited as a solution of polycarbonate polyurethane dissolved in Hexafluoroisopropanol (HFIP) (DuPont, Wilmington, DE, USA) at 12% w/v. The electrospinning apparatus used was commercially available from IMfE Technologies, Geldrop, Netherlands. The electrospun fibers were collected on a target aluminum mandrel rotating at 800 rpm and placed at a distance of 22 mm from the syringe tip to deposit an isotropic fiber to produce a structural support having an average wall thickness of 500 microns. The structural supports were dried in a vacuum to remove residual solvent. The structural supports were then plasma treated with 2 consequent cycles of ethylene and oxygen gases using a low-pressure plasma system (Diener Tetra 150-LF-PC-D). Structural supports were gamma sterilized (STERIS, Northborough, MA). The applied dose range was 25-35 KGy.

The resulting tubes were polymeric structural supports composed of electrospun polyurethane having a consistent outer diameter (OD) of 22 mm and a length of 11 cm.

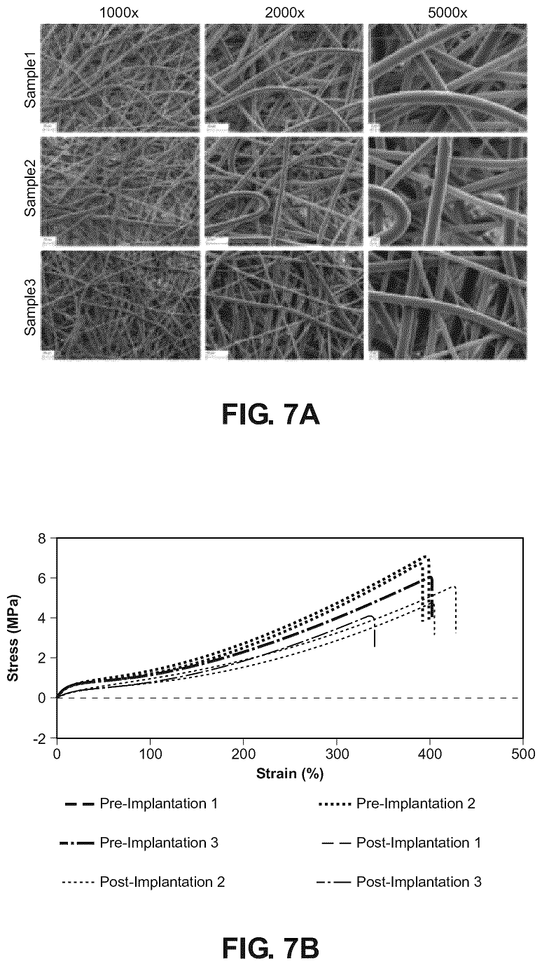

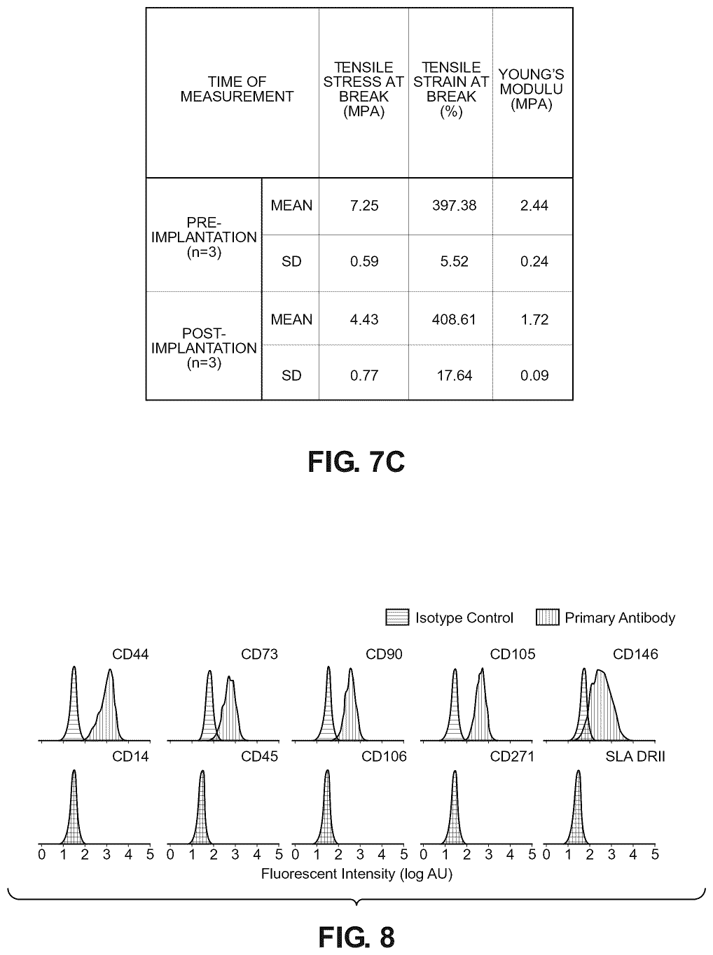

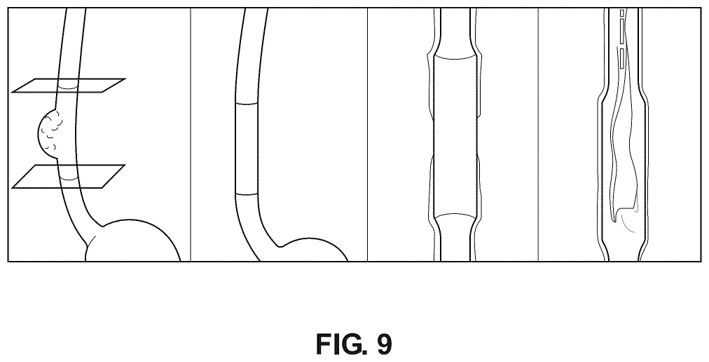

The morphology of the electrospun fibers was analyzed by scanning electron microscopy (Zeiss-EVO MA10). Samples of the structural supports were sputter coated with Platinum and Palladium using a sputter coater for two minutes (Cressington-208HR, TED PELLA, Inc, Redding, CA) under a pressure of 8×10² mbar and an electric potential of 300 V. Porosity was calculated using gravimetric measurements. Porosity, ε, is defined in terms of the apparent density of the fiber mat, ρAPP and bulk density of the polymer, ρPU of which it is made: ε=1−ρAPP/ρPU. The apparent structural support density ρAPP was measured as mass to volume ratio on 10 mm dry disks: ρAPP=Mass/VPU. Pore size measurements were taken using a mercury porosimeter system (Micromeritics AutoPore IV). Tensile tests on were performed consistent with ASTM D638 guidelines on 10 mm×40 mm samples that were mounted on an electromechanical load frame (Instron 5943 Apparatus) using a 1 kN load cell. The testing parameters were the same for all samples, at a 100 Hz data acquisition rate, a gauge length of 30 mm, and a test speed of 1 mm/sec. Scanning electron microscopy at increasing magnifications as illustrated in FIG. 7A demonstrated the isotropic fiber arrangement aspects of the electrospun synthetic structural support. The smooth surface and isotropic nature of the fibers ensures strength and elasticity of the structural support is uniform in all directions.

Tensile testing via uniaxial mechanical loading was performed on three pre-implantation and three post-implantation structural supports (FIG. 7B), which all showed similar results at in vivo loading values. Consistency between the six samples at in vivo loading shows that the structural supports have a low degree of variability present after fabrication and in vivo implantation (FIG. 7B, C). The mean (±SD) tensile strain ranged between 119.5±1.61 mm and 124.5±3.44 mm across the six structural supports. At failure, the tensile strain for the samples at pre-implantation reached 397.38% 5.52% and post-implantation 408.61%±17.64%. Strain values above 400% suggest the reliability of the fabrication process and relative in vivo stability. Tensile stress at failure was 7.25±0.59 MPa and 4.43±0.77 MPa for pre- and post-implantation structural supports, respectively. Consequently, the Young's modulus was larger in the pre-implantation samples than the post-implantation samples, though both groups were comparable in elasticity at in vivo strains (FIG. 7B, C). The load at failure followed the same trend as the Young's modulus, with the pre-implantation values being greater than the post-implantation values.

Autologous porcine adipose-derived mesenchymal stem cells (aMSCs) were isolated from 8 pigs following an open adipose biopsy and analyzed for characterization. The 8 Yucatan mini-pigs underwent general anesthesia and chlorhexidine skin preparation prior to a sterile, open adipose tissue biopsy taken from the lateral abdominal wall. A 5 cm incision was performed next to the linea alba with hemostasis achieved using electrocautery. Approximately 30-50 g of adipose tissue was isolated, and transferred to a 50 mL conical tube containing alpha Minimal Essential Medium (MEM)/glutamax (Thermo Fisher Scientific, Waltham, MA) and 1% penicillin/streptomycin (Thermo Fisher Scientific).

20-60 g of abdominal adipose tissue was surgically excised from each anesthetized Yucatan mini pig (50-60 kg body weight). The tissue samples were washed 3 times in alpha Minimal Essential Medium (MEM)/glutamax (Thermo Fisher Scientific) and 1% penicillin/streptomycin (Thermo Fisher Scientific). The washed tissue was trimmed to remove lymph nodes and blood vessels and minced into pieces smaller than 5 mm. The tissue pieces were dissociated in digestion buffer (300 IU/mL collagenase type II, 0.1% bovine serum albumin (7.5%, fraction V), 1% penicillin/streptomycin, alpha MEM/glutamax) for 55 minutes at 37° C., 5% CO₂. After quenching in complete growth medium (StemXVivo, R&D Systems, Minneapolis, MN) and 1% penicillin/streptomycin), the cells were centrifuged for 15 minutes at 1500 rpm. The cell pellet was re-suspended in 5 mL of growth medium and filtered through a 70 μm filter. The cell filtrate was centrifuged for 5 minutes at 1500 rpm. The cell pellet was re-suspended in 5 mL of growth medium and cells were plated according to tissue weight (3 g of adipose tissue isolate per T75 flask containing 20 mL growth medium).

Cells were washed twice in PBS without calcium or magnesium (Thermo Fisher Scientific) and dissociated using TrypLe (Thermo Fisher Scientific). The dissociation was quenched with growth medium, and the cells were centrifuged at 1000 rpm for 5 minutes. The cell pellet was re-suspended in 1% bovine serum albumin diluted with PBS. Aliquots of 1 million cells were incubated in antibody at 4° C. for 30 minutes in the dark (Supplemental Table 1). The labeled cells were washed 3 times in buffer and secondary antibodies (Life Technologies, Carlsbad, CA) were applied as necessary at 4° C. for 30 minutes in the dark. After a further 3 washes, the cell suspensions were placed into a 96 well plate for flow cytometry (Guava easyCyte HT, EMD Millipore, Billerica, MA). Events representative of live cells were gated on forward and side scatter values, based upon measurements of viability (ViaCount, EMD Millipore). Cell type analysis was performed using fluorescent events compensated against unstained and isotype control antibody-stained samples. Acquired data was exported and analyzed using standalone software (FlowJo version 10, FlowJo, LLC, Ashland, OR).

To assess colony formation, adipose-derived cells were isolated as described, triturated to a single cell suspension and diluted to 10 cells/mL of growth medium. 100 μL of the cell suspension was added to each well of a 96 well plate (Corning, Inc., Corning, NY) and visually inspected for cell number the following day. After 5-7 days, colonies of cells became visible and medium was changed every 3 days until the colonies contained at least 50 cells. Wells were counted for the presence of colonies and expressed as a percentage of total wells analyzed.

Pluripotency of isolated adipose-derived cells were determined by their ability to undergo adipogenesis and osteogenesis by chemical induction. Cells were plated in 6-well tissue-culture plates, cultured in complete growth medium, and allowed to grow to 60% or 100% confluency for adipogenic and osteogenic differentiation, respectively. Upon reaching confluence, medium was changed to either adipogenic or osteogenic differentiation medium (CCM007, R&D Systems, Minneapolis, MN). Medium was changed every 2 days until 14 days in culture. Cells cultured in adipogenic differentiation medium were stained with Oil Red O (American MasterTech, Lodi, CA) to identify lipids and cells cultured in osteogenic medium were stained with Alizarin Red (EMD Millipore) for calcium deposition.

Concentrations of glucose and lactate were measured in conditioned medium from bioreactors at the time of seeding and 2, 5, and 7 days post-seeding (iSTAT, Abbott, Princeton, NJ).

Cell supernatants were analyzed for the production of porcine cytokines and growth factors either by multiplex assay on the Luminex 200 platform or by ELISA at the University of Minnesota Cytokine Reference Laboratory using commercially available kits and performed according to manufacturers' directions. A 13-plex porcine-specific bead-set panel (EMD Millipore) was used to determine levels of porcine VEGF, GM-CSF, IL-1RA, IL-6 and IL-8. Values were interpolated from standard curves generated on each plate using BioPlex software (BioRad, Hercules, CA) for the Luminex platform, or Microplate Manager software for ELISA plates read on a BioRad 550 plate reader. All samples were assayed in duplicate.

Cells were rinsed in PBS and fixed with 10% formalin for 15 minutes at room temperature. The cells were gently rinsed 3 times in PBS containing 0.1% Triton X-100 (PBS-T) and incubated for 1 hour at room temperature in 10% normal goat serum (Vector) diluted in PBS-T. The rabbit anti-nestin antibody (Biolegend, 1:100) was diluted in 10% normal goat serum and PBS-T and incubated overnight at 4° C. The cells were rinsed twice in PBS-T and incubated in fluorescent goat anti-rabbit antibody (Alexa Fluor 594, Thermo Fisher Scientific) at room temperature for 1 hour. The cells were rinsed twice and counterstained with 4′,6-diamidino-2-phenylindole (DAPI).

After 48 hours at 37° C., the cells were washed twice in phosphate buffered saline containing calcium and magnesium (Thermo Fisher Scientific) and replaced with fresh growth medium. Thereafter, culture medium was replaced every 2 days until the flasks were 70%-80% confluent. At passaging, the cells were dissociated (TrypLe, Thermo Fisher Scientific), counted (Countess, Thermo Fisher Scientific) and replated at 200,000 cells per T175 flask. The cells were passaged twice prior to seeding of structural supports.

Each 11 cm long structural support was placed in a bioreactor and seeded with 32 million cells (viability >70%, trypan blue dye exclusion, Countess, Thermo Fisher Scientific) in growth medium supplemented with 0.1875% sodium bicarbonate (Thermo Fisher Scientific), MEM eagle (Lonza) and 1.19 mg/mL bovine collagen (Organogenesis) in 0.01M hydrochloric acid. The cells were incubated for 5 minutes at 37° C., 5% CO₂ before 200 mL of growth medium was slowly added to the bioreactor. The bioreactor was incubated for 7-8 days prior to structural support implantation. Culture media was changed every 2 days and taken for various assays described below.

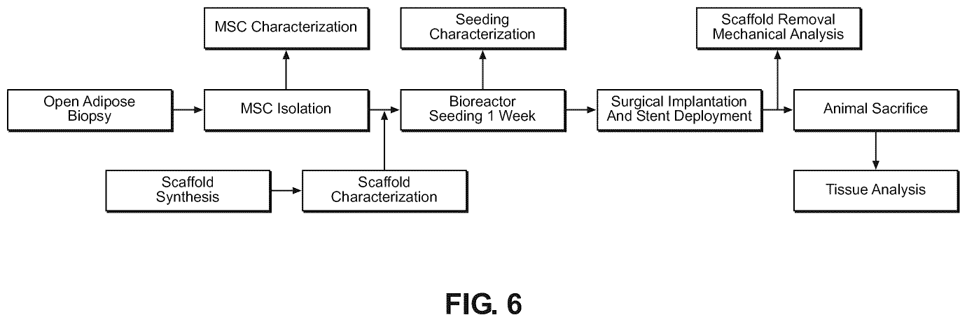

The porcine aMSCs were seeded onto a previously characterized structural support and subsequently incubated in a bioreactor. Seeded structural supports were then implanted following esophagus resection in Yucatan mini-pigs until structural support removal at 3 weeks (FIG. 6) and reproducibly stained positive for known MSC markers using anti-porcine CD44, CD73, CD90, CD105, and CD146, antibodies and were negative for CD14, CD45, CD106, CD271, and SLA Class II DR. Greater than 95% of the cultured cells stained positive for nestin and aSMA, indicating stem cell characteristics are maintained in culture. Pluripotency was determined by chemically inducing the porcine MSC isolates to undergo adipogenesis and osteogenesis, respectively. These aMSCs were routinely expanded and characterized from passage 1 to 5 and showed consistent phenotypic and functional characteristics.

Porcine aMSCs grown from passage 2 were seeded onto a polymeric structural support and incubated in a bioreactor for 7 days (+/−1 day) at 37° C. A number of cytokines and growth factors were measured using enzyme-linked immunosorbent assay (ELISA) to determine if the seeded aMSCs cultured on the structural support secrete factors that may assist in angiogenesis and immunomodulation. Cell secretion of vascular endothelial growth factor (VEGF), granulocyte-macrophage colony-stimulating factor (GM-CSF), interleukin (IL)-6, IL-8, and IL-1RA was detected in conditioned medium at levels significantly above medium alone (FIG. 4A). However, additional cytokines, TNF-α, IL-1α, IL-1β, INF-γ, IL-10, IL-12, IL-18, platelet-derived growth factor (PDGF), and regulated on activation, normal T expressed and secreted (RANTES), were measured but not detected.

Punch biopsies of sections of the seeded graft were taken at the end of the incubation time at 7 days, to assess cell health and penetration into the structural support. Cellular health was assessed by immunofluorescence staining using calcein (live cells) and ethidium bromide (dead cells). Cellular penetration of the structural support was assessed using ethidium bromide for cell identification. The populations of live cells attached to the structural support are indicated by the predominance of calcein staining of the biopsy samples. On cross sections of the structural support biopsies the majority of cellular attachment was present at the surface of the structural support. While there was some evidence of cellular proliferation and ingrowth within the structural support. Metabolic activity of the implant graft during bioreactor incubation was measured every 48 hours for glucose uptake and lactate production. Measurements of conditioned medium consistently indicated decreased glucose and increased lactate levels over time, both indicators of continued metabolic cell growth. In addition, cell expansion over 7 days in the bioreactor was quantified by total DNA content which increase several fold over the course of bioreactor cell seeding. Further characterization of cell phenotype on the structural support following 7 days incubation shows that the cells continue to express alpha smooth muscle actin (aSMA) and nestin.

After endotracheal intubation and induction of general anesthesia, animals were placed in a left lateral decubitus position. Hair was clipped and Chlorhexidine or povidone iodine was used for skin preparation and the animal was sterilely draped. A standard right thoracotomy at the level of the 4^{th} intercostal space on each animal was performed and the thoracic cavity was entered. Single lung ventilation was achieved through the use of a double lumen endotracheal tube. A 4-4.5 cm segment of the esophagus, located in the mid thoracic region (posterior to the right lung hilum, was circumferentially mobilized and resected to generate a 6 cm defect (tissue retraction proximally and distally). The seeded structural support (6 cm length) was then implanted using polydioxanone (PDS, Ethicon Inc., Somerville, NJ) absorbable sutures with anastomosis to the proximal and distal esophagus. After the implantation, a commercially available esophageal stent (WallFlex M00516740, Boston Scientific) was inserted under direct endoscopic guidance (Storz Video Gastroscope Silver Scope 9.3MM×110CM, Tuttlingen, Germany). Stent deployment was performed under endoscopic and surgical visualization. The esophageal stent was fixed in place to the normal esophageal tissue using absorbable suture, at both the proximal and distal stent flares.

Postoperatively the animals were adjunct supported by gastrostomy feeding and maintained on a liquid diet through a feeding tube for 2 weeks, a mashed diet for a period of 2 more weeks, and then allowed to eat an oral diet of solid food after for the continuation of the study.

At approximately 21 days following the implantation, the structural supports were retrieved endoscopically and aMSC impregnated platelet rich plasma (PRP) gel was applied to improve the healing process of the newly formed esophageal conduit. After PRP application, a new fully covered esophageal stent (WallFlex™, 12 cm long×23 mm outer diameter, Boston Scientific Corporation) was placed across the implant zone to prevent stricture formation and to maintain anatomy during regeneration. Every two weeks the animals underwent sedation and assessment of the esophageal anastomosis and esophageal stent exchange to allow direct visualization and progression of esophageal regeneration. Follow-up observations were conducted endoscopically (Storz Video Gastroscope Silver Scope 9.3MM×110CM, Tuttlingen, Germany).

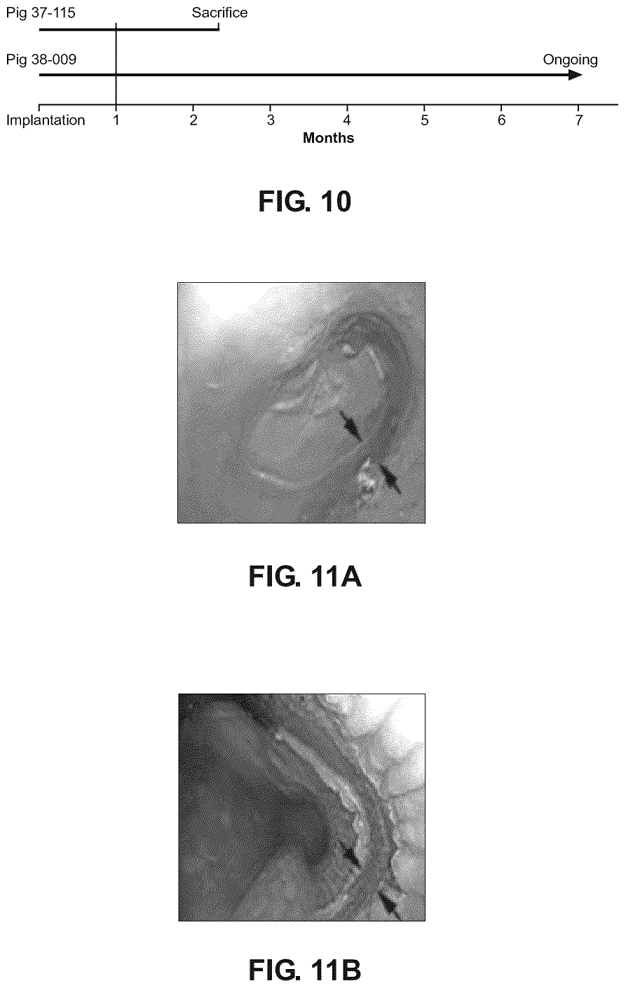

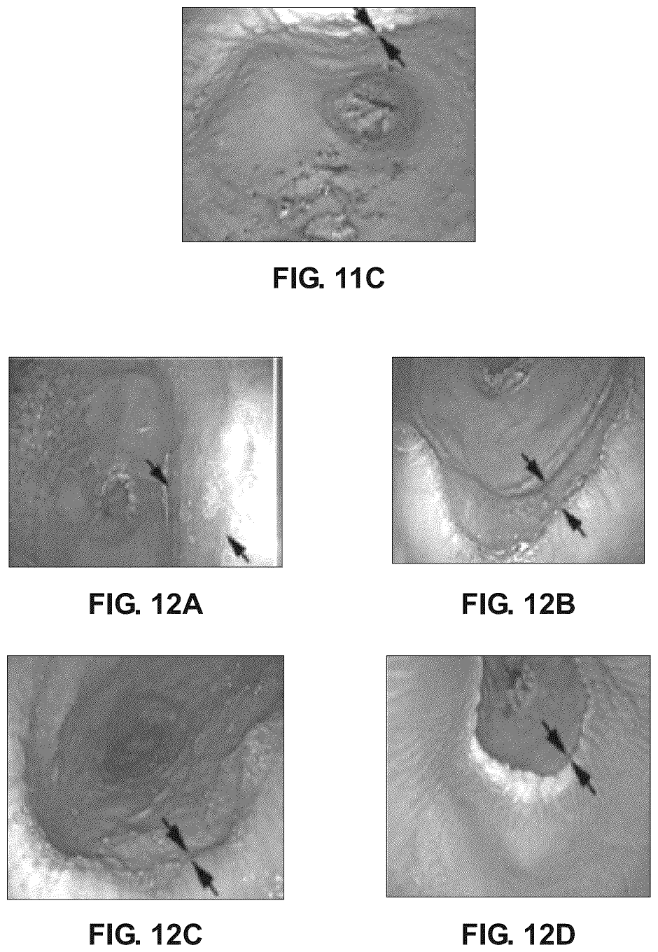

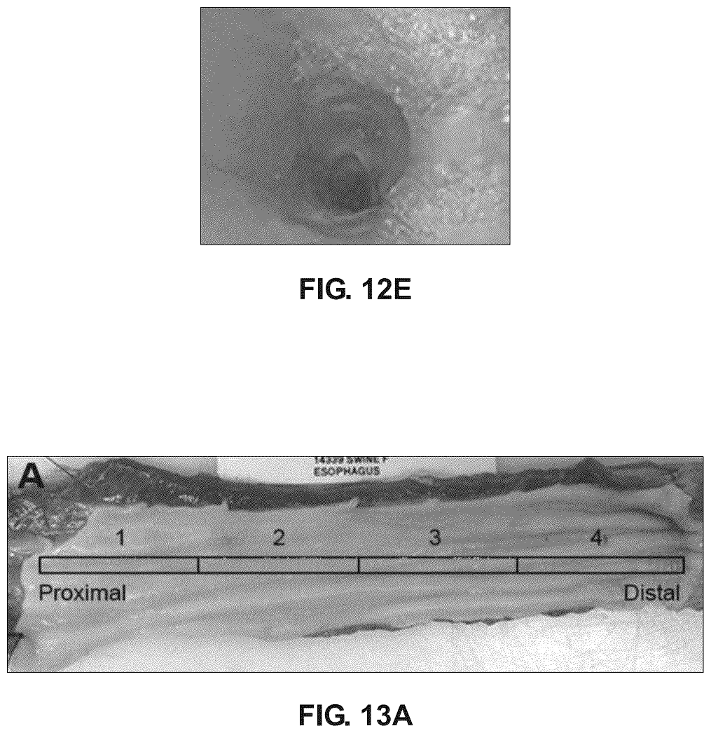

Regeneration progression was also assessed by endoscopic inspection. Following structural support removal. the implant zone was visualized endoscopically at approximately 3-4 week intervals; 2 representative animals are shown (FIGS. 10 and 11). At 3-4 weeks post-implantation, regeneration of the mucosal layer was only partially complete. However, the process of esophageal healing continued with time, indicated by the proximal and distal ends of the mucosal layers forming an initial ridge before fusion of the 2 layers and complete mucosal regeneration. The early reconstitution of the esophageal continuity and integrity and the subsequent growth of the submucosa from the two opposite edges of the resection have been consistent across all eight animals; 2 animals have been maintained to 8 and 9 months post-surgery and have been without esophageal stent respectively for 2 and 3 months without evidence of stricture or stenosis and have had durable oral intake, with noteworthy weight gain.

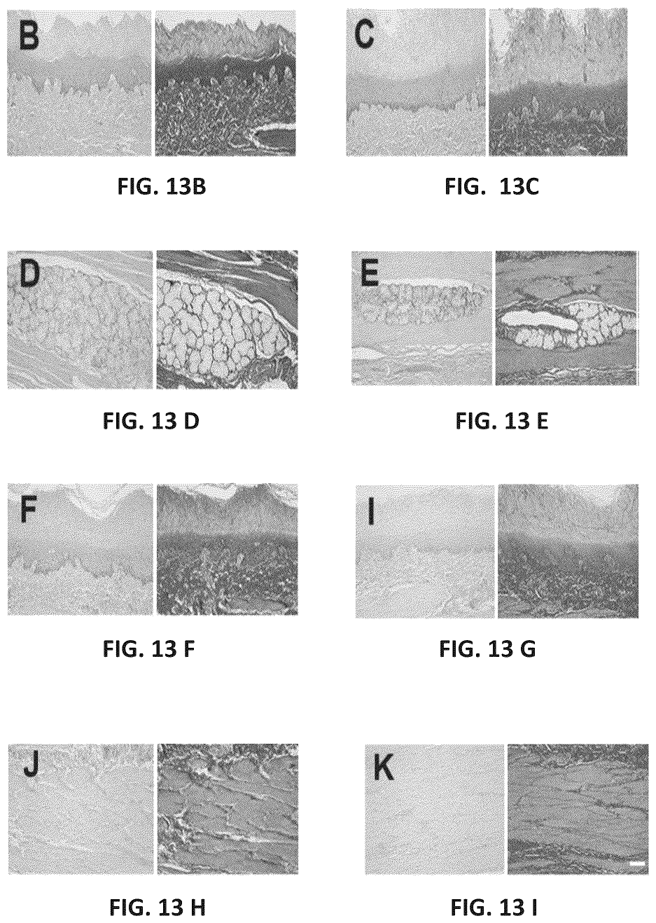

In order to ascertain histological similarities of the morphologies of regenerated and native esophageal tissue. Samples of tissue were excised from a representative pig esophagus at 2.5 months post-implantation and include both the site of surgery and adjacent distal and proximal tissues for histology. (FIG. 13A, dotted box indicates the histological analysis specimens). Representative images of hematoxylin and eosin (FIGS. 13B and D) and Masson's trichrome (FIGS. 13C and E) stained tissue sections show histologically intact multi-layered esophageal epithelia and submucosa and normal inner muscular layer morphology.

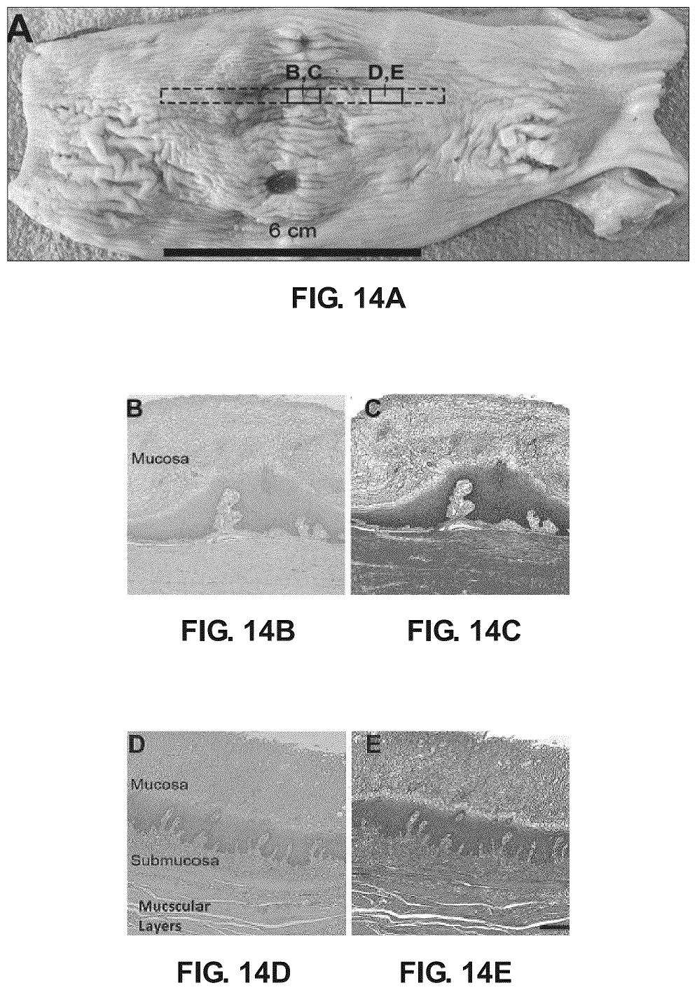

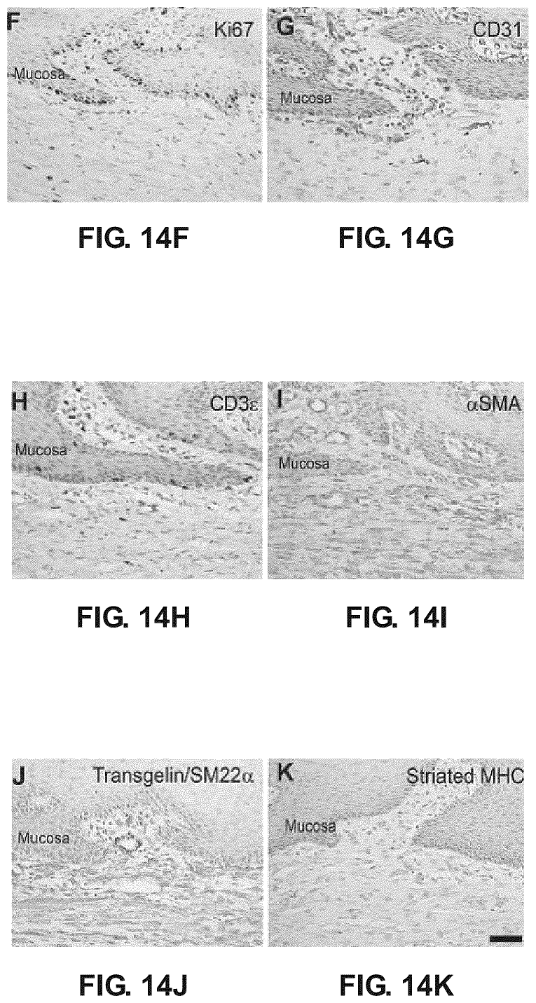

Representative immunohistochemical analysis from the regenerated region is depicted in FIG. 14 which depicted histological analysis of tissue from pig esophagus at 2.5 months post implantation of a cellularized structural support as described herein. FIG. 14 A depicts a macroscopic image of excised esophagus (proximal suture to the left). Samples of tissue were excised to include the site of surgery, monitored by endoscopy, with adjacent distal and proximal tissues for histology (dotted box). (FIG. 14 B-E) Representative images of hematoxylin and eosin (FIG. 14 B, D) and Masson's trichrome (FIG. 14 C, E) stained tissue sections. Scale bars: A=6 cm, B, C, D and E=200 μm. Representative immunohistochemical analysis demonstrates immunoreactivity for Ki67 (FIG. 14F) suggesting continued proliferation of mucosal and submucosal cells, CD31 (FIG. 14G), CD3e (FIG. 14H), aSMA (FIG. 14I), transgelin/SM22a (FIG. 14J) and a relative absence of striated myosin heavy chain (K) in tissue at the site of surgery. Scale bars: F-K=200 μm demonstrates immunoreactivity for Ki67 (FIG. 14F) at 2.5 months suggests continued proliferation of mucosal and submucosal cells, CD31 (FIG. 14G), CD3e (FIG. 7H), aSMA (FIG. 14I), transgelin/SM22a (FIG. 14J) and a relative absence of striated myosin heavy chain (FIG. 14K) in tissue at the site of surgery. The predominance of aSMA, SM22a, and relative absence of myosin heavy chain suggest that smooth muscle proliferation precedes skeletal muscle growth.

The synthetic matrix seeded with autologously derived mesenchymal cells (aMSCs) resulted in full longitudinal regeneration of the resected esophagus with minimal mucosal ulcerations or perforations (Table 1). All animal experienced a full 100% of longitudinal regeneration from 2-9 weeks after graft removal with 1 out of 6 animals experiencing both mucosal ulceration or perforation. No animals experienced leaks over the course of the study.

TABLE I

| | | | Structural | Longitudinal | | | |
|---|---|---|---|---|---|---|---|
| | Time | | support length | regeneration | Mucosal | Contained | |
| Pig No | (status) | Stent | (cm) | (%) | ulceration | perforation | Leak |
| 1 | 2 weeks | No | 4.5 | 100 | No | No | |
| | (euthanized) | | | | | | |
| 2 | 2 weeks | No | 4.5 | 100 | No | No | |
| | (euthanized) | | | | | | |
| 3 | 6 weeks | Yes | 6 | 100 | No | No | |
| | (euthanized) | | | | | | |
| 4 | 7 weeks | Yes | 6 | 100 | No | No | |
| | (euthanized) | | | | | | |
| 5 | 9 weeks | Yes | 6 | 100 | No | Yes | |
| | (euthanized) | | | | | | |
| 6 | 9 weeks | Yes | 6 | 100 | Yes | No | |
| | (euthanized) | | | | | | |
| 7 | 7 months (alive ) | Yes | 6 | | | | |
| 8 | 7 months (alive ) | Yes | 6 | | | | |

### Example III—Other Gastrointestinal Implant

The process as outlined in Examples I and II is implemented replacing gastrointestinal regions localized to the rectum. Results are similar to the results outlined previously.

### Example IV—Long Gap Atresia Repair

In order to simulate repair of Long Gap atresia, pre-clinical porcine studies were conducted in which a large portion of native esophagus was resected and replaced with the synthetic structural support device described in Example II, using an end-end anastomosis. At 21 days post-implantation, the structural support component which did not integrate into the developing tissue was endoscopically removed, revealing a continuous, regenerated, fibrovascular tube of tissue. Endoscopic evaluation of the lumen revealed the development of a mucosal epithelial layer across the implant that was fully formed by 90 days postimplant. Functionally the animals were able to eat and gain weight. Macroscopic and microscopic histologic analyses at multiple necropsy time points post-surgery revealed the early formation of fibrovascular neo-tissue followed by epithelialization of the lumenal surface. Nine-month post-surgical endoscopy analyses also demonstrated an intact epithelialized lumen.

A similar strategy for esophageal reconstruction was tested in a porcine model, where autologous bone marrow MSCs (BM-MSC) were seeded onto an acellular small intestine submucosal construct. The results from this study demonstrated reepithelialization as well as the development of muscular structures in animals surviving beyond 50 days post-implant with the longest survival time of 119 days. However, this study repaired the abdominal esophagus and used 2 surgical procedures, an initial surgery to “maturate” the construct in the momentum and then a second surgery to repair a 3 cm full circumferential resection with the maturated tissue engineered tube.

Twelve animals were implanted and recovered from surgery. Two early terminations took place in the 90-day cohort. One animal was euthanized one day after surgery due to hindlimb paralysis and another animal was euthanized 12 days after surgery due to pericardial effusion and lung disease. Autopsy pathology indicated that the hindlimb paralysis was not caused by surgery and the lung disease appeared to be present well before surgery. The other early termination was in the 365-day cohort at Day 297 due to dehydration, vomiting, and decreased body condition associated with an incidental small intestine volvulus. The results summarized in Table II indicate that the use of seeded synthetic structural support was safe and feasible.

TABLE II

| | Cohort design | | | | | |
|---|---|---|---|---|---|---|
| | animal numbers | | | | | |
| | per group | | Survival | | | |
| Study | | Surgical | to term | | Early deaths | |
| duration (Days) | Test | Control | Test | Control | Test | Control |
| Cohort 3 | 4 | 1 | 4 | 1 | 0 | 0 |
| 30 ± 3 days | | | | | | |
| Cohort 2 | 5 | 1 | 3 | 1 | 1 @ D1 | 0 |
| 90 ± 3 days | | | | | 1 @ D12 | |
| Cohort 1 | 3 | 1 | 2 | 1 | 1 @ D297 | 0 |
| 365 ± 15 days | | | | | | |

Study design matrix indicating study duration (cohort), numbers of animals per cohort and survival times for test (AD-MSC seeded scaffold implanted) and control animals (surgical esophagectomy with primary anastomosis).
D1 day 1, D12 day 12, D 297 day 297.

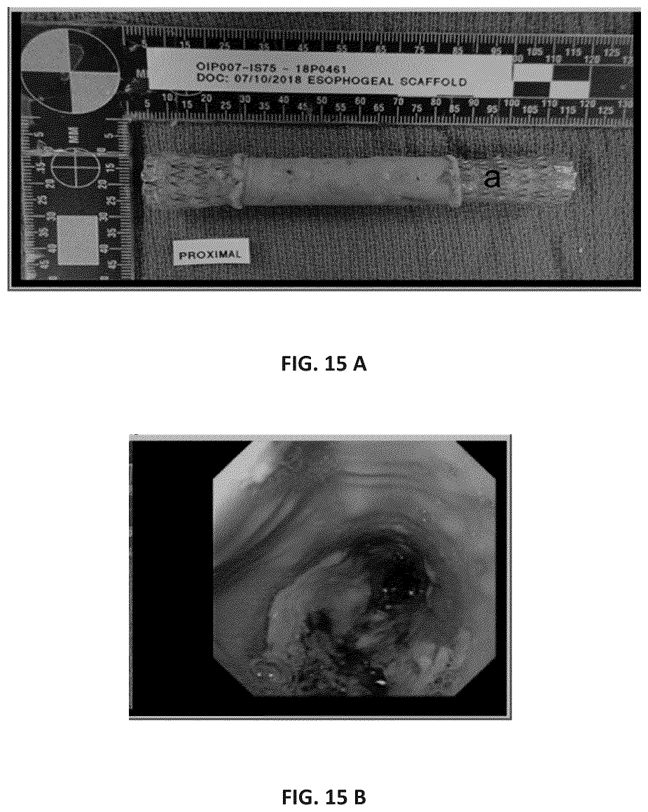

In surgical control and test animals, the initial stent was removed endoscopically at day 21 post surgery. In the synthetic structural support test recipients, the structural support component of the synthetic structural support was also removed as it adhered to the stent and released from the neo-tissue. (See FIG. 15 A). Endoscopic visualization of the newly formed adventitial tissue revealed a complete contiguous tube that spanned the implant site.

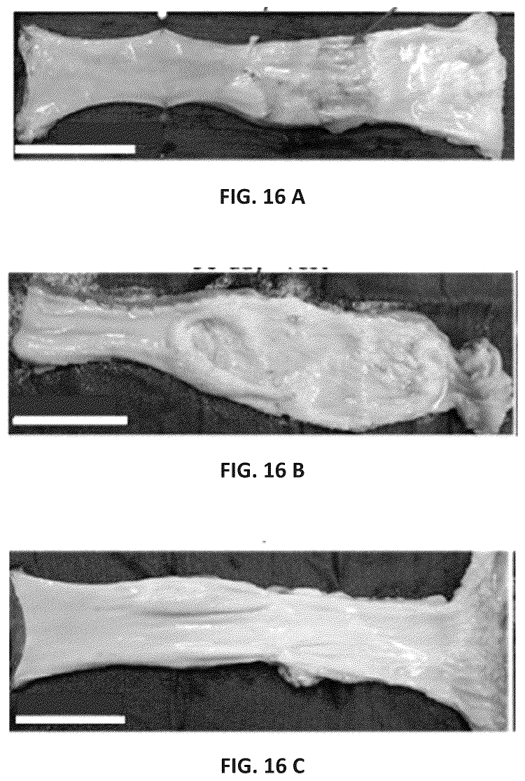

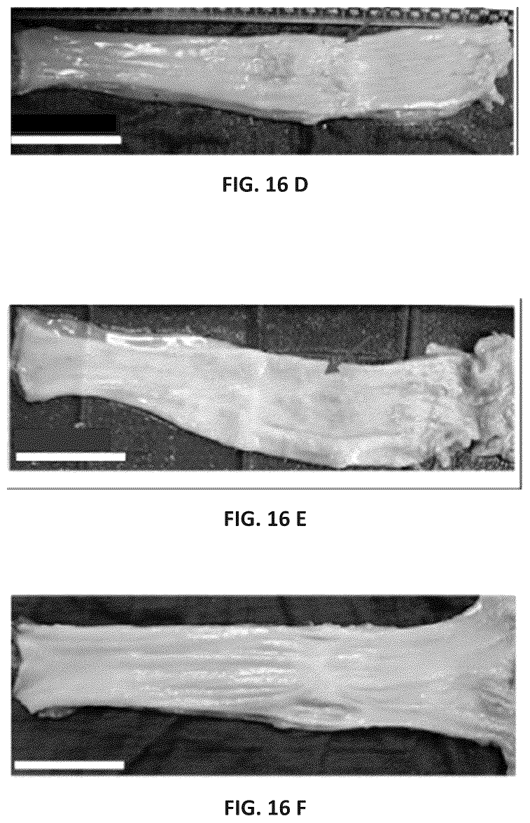

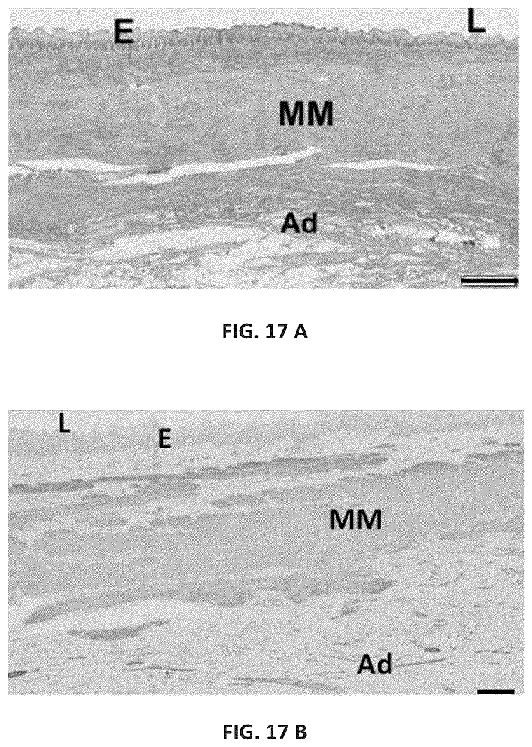

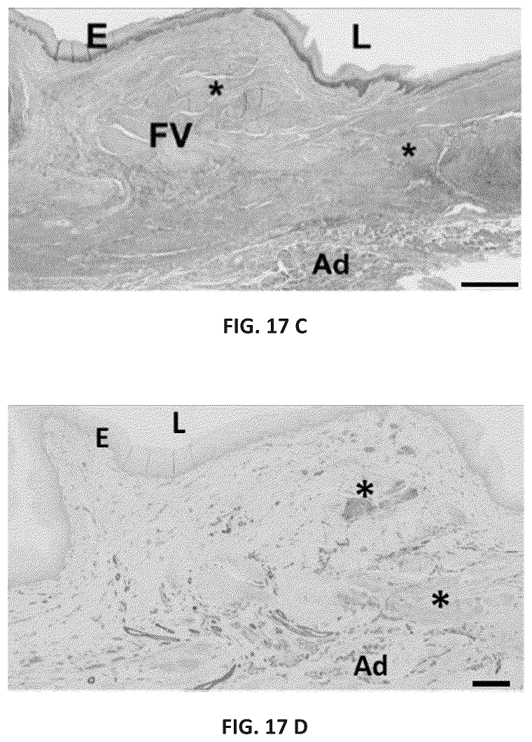

All animals were re-stented and returned to their pens. The 30-day cohort was euthanized at 9 days post resenting and underwent necropsy analyses revealing an intact tubular structure with un-epithelialized lumen in both the surgical control group and in the synthetic support structure cohort. The tissue at the implant site appeared red and unepithelialized as indicated by red arrows. See FIGS. 16 A, 16 D and 17 A and 17 B. In FIGS. 17A to 17D. E=epithelium; L=lumen; FV=fibrovascular tissue; Ad=adventitia; asterisks indicate areas of muscle cell staining in C, D). Scale bars=1 mm in the MT stained slides (A, C) and 500 μm in the SM22 stained slides (B, D).

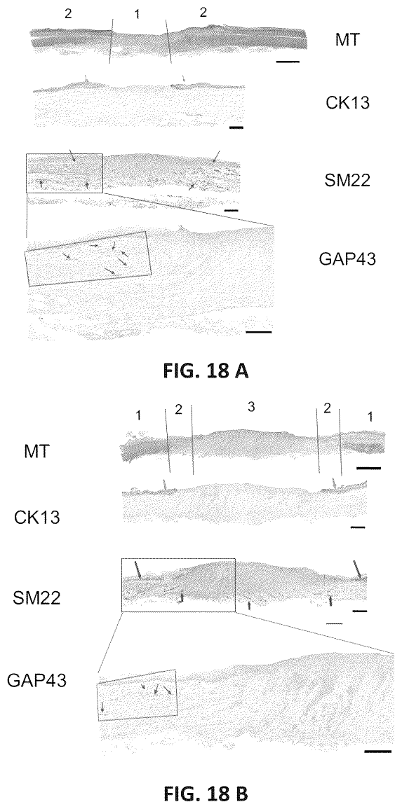

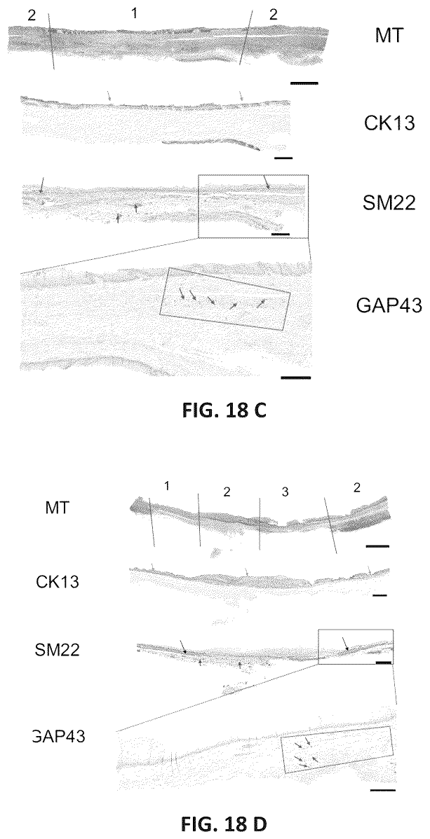

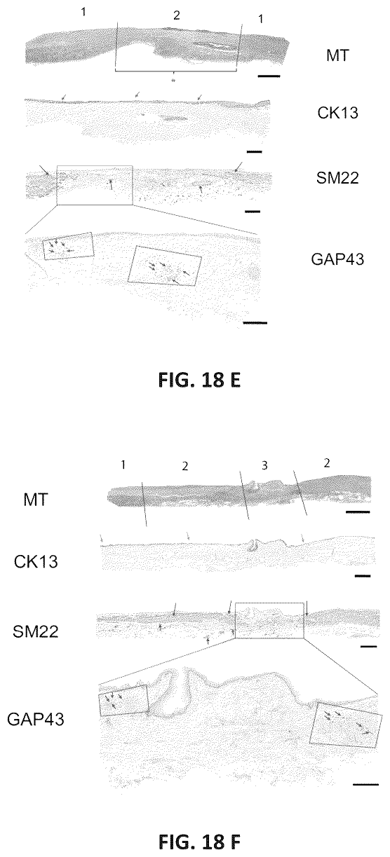

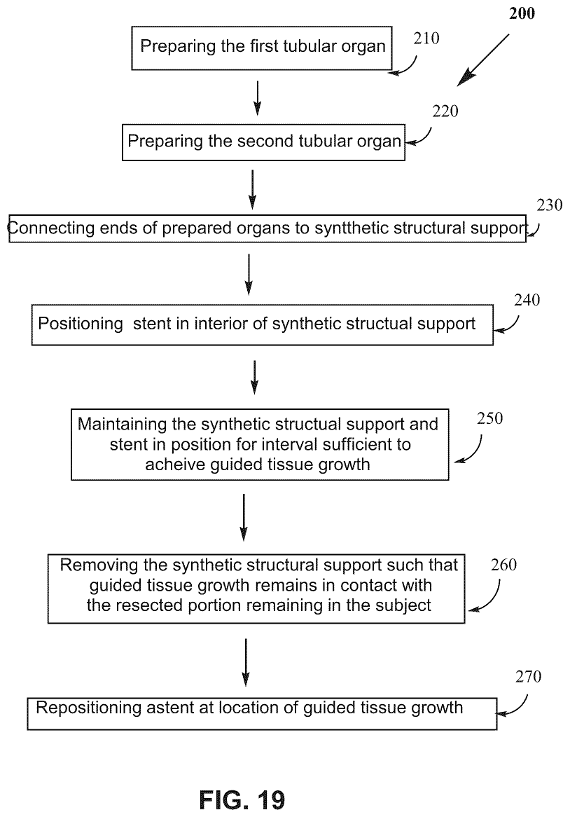

Epithelialization was complete by 3 months post-implantation in the both the synthetic support structure implant and the control groups as determined by endoscopy, gross histology, and MT histology. See FIGS. 18 A and B and 16 B-F and 17 C-F respectively. Stents were utilized and on average were removed by day 120 in the 365-day cohort. Stents in the 30-day and 90-day cohorts were utilized throughout the in-life period and served to maintain the position of the synthetic structural support.

The initial stents were deployed immediately after surgery and subsequently removed at day 21 post-implantation. To accommodate for growth of the animal, the stents were exchanged every 3-4 weeks with increasing diameter sizes unless symptoms of migration or obstructions presented sooner. To visualize the esophageal stent, an endoscope with live video feed was used in conjunction with fluoroscopy. If the esophageal stent was removed, the esophagus was visualized after removal to determine if the esophagus sustained any injury during the process as well as to monitor regeneration. A new stent was then inserted and deployed under fluoroscopic and endoscopic guidance. For Cohort 1, stents were permanently discontinued once the mucosal layer was fully formed and the maximum stent diameter of 23 mm was reached (between 3 and 6 months post-implant). For Cohorts 2 and 3, the stents were utilized until termination. The explanted esophageal tissue was further studied to assess fibrotic scar tissue formation using Mason's trichrome (MT) stain and immunohistochemical analysis (cytokeratine-13, CK13; smooth muscle transgelin, SM22; and growth associated protein-43, GAP43) of excised esophagus from the three surgical control animals (CNTL: at 30 days in FIG. 18 A; at 90 days in FIG. 18 C; and at 365 days in FIG. 18 E) and in animals implanted with the synthetic structural support as described in Example I (at 30 days in FIG. 18 B; at 90 days in FIG. 18 D; at 365 days in FIG. 18 F). Scale bars in the MT=4 mm. Scale bars in the CK13, SM22, and GAP43 panels=2 mm

In each of FIGS. 18 A-F, the top panel stained with MT presents the resected area of the surgical controls (CNTL) and the implant regions of the CEI recipient animals at 30-days, 90-days, and 365-days post-surgery. Tissue Zones are labeled and include Zone 1—native tissue flanking the resection; Zone 2—transition zone; and Zone 3—central fibrovascular tissue evident in FIGS. 18 B, 18 D and 18 F.

Cytokeratin (CK13) immunohistochemistry (IHC) illustrates the cytokeratin positive epithelial layer spanning the resection (green arrows) in the synthetic structural support implant. SM22 IHC identifies the smooth muscle components of the native tissue and in the regenerating regions in the synthetic structural support implant recipients (note arrows). SM22 also identifies the vascular structures throughout the resected area and within the synthetic structural support implant zones (red arrows). GAP43 IHC identified growth cone positive neuronal structures at the border of the resected area and at the proliferative fronts of the regenerating smooth muscle tissue migrating into the synthetic structural support implant zones (note arrows with the boxes). Boxed region in the SM22 IHC panel represents the area in the GAP43 IHC panels. In FIG. 18 E, the bracket labeled with an asterisk indicates the area of the resection in the surgical control that exhibits persistent non-regenerating fibrovascular tissue across zone 2 at 1-year post-surgery evidencing the presence of fibrotic scar tissue. That is not in evidence in explanted tissue from synthetic support structure implant zones.

Having thus described several embodiments with respect to aspects of the inventions, it is to be appreciated various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for reducing remodeling formation of fibrotic tissue in a tubular organ comprising the steps of:
resecting a portion of a tubular organ in a subject producing a resected organ portion, the resected organ portion remaining in the subject;
removing a portion of the tubular organ from the subject
replacing the removed portion with a synthetic support structure at an implantation site, the synthetic support structure having a first end, a second end opposed to the first end and middle section extending between the first end and the second end, wherein at least a portion of the synthetic support structure is configured as a tubular member defining an interior lumen, the synthetic support structure further having an outer polymeric surface extending from the first end to the second end, and a cellularized sheath layer adhering to at least a portion of the outer polymeric surface;
maintaining the first end of the synthetic support structure in direct contact and sutured to a distal section of the tubular organ creating a first tubular organ-synthetic support anastomosis;
maintaining the second end of the synthetic support structure in direct contact and sutured to a second section of the tubular organ creating a first tubular organ-synthetic support anastomosis, wherein the first end and the second end are maintained in contact for a period of time sufficient to achieve guided tissue growth along the synthetic support structure the guided tissue growth derived from and in contact with the tissue present in the resected organ portion remaining in the subject, the guided tissue growth occurring around the synthetic support structure; and
after achieving guided tissue growth, removing the synthetic support structure from the implantation site, the step of removing the circumferential portion of the tubular organ occurring in a manner such that the guided tissue growth is continuous and remains in the contact with the resected portion of the tubular organ remaining in the subject.

2. The method of claim 1 further comprising:
imparting cellular material onto the polymer surface of the synthetic support structure; and
allowing the cellular material to grow to form the cellular sheath layer, the imparting and allowing steps occurring prior to the step of resecting the portion of the tubular organ.

3. The method of claim 2 wherein the synthetic structural support is a tubular member and wherein the outer polymeric surface includes outwardly positioned electrospun polymeric fibers.

4. The method of claim 3 wherein the cellularized sheath layer spans at least a portion outwardly positioned electrospun fibers.

5. The method of claim 1 wherein the cellularized sheath layer is composed of cellular material, the cellular material including at least one of mesenchymal cells, stem cells, pluripotent cells.

6. The method of claim 1 wherein the tubular organ is a gastrointestinal organ structure that includes an esophagus, a stomach or a combination of an esophagus and a stomach.

7. The method of claim 1 wherein the removing step is achieved intrascopically.

8. The method of claim 2, comprising:
resecting a portion of a tubular organ in a subject producing a resected organ portion, the resected organ portion remaining in the subject and having a resection edge and forming a resection site;
implanting a synthetic support structure at the resection site, the synthetic structural support having an outer polymeric surface and including a first end and a second end opposed to the first end, an outer polymeric surface positioned between the first end and the second end and a cellularized sheath layer overlying at least a portion of the outer polymeric surface, wherein at least a portion of the celluralized sheath layer is proximate to the resection edge of the resected organ portion; and
maintaining contact between the synthetic support structure and the resection edge for an interval sufficient to achieve guided tissue growth along the synthetic support structure,
wherein at least a portion of the synthetic support structure is absorbed at the resection site within a period of time sufficient to achieve guided tissue growth along the synthetic support structure.

9. The method of claim 8 wherein the synthetic support structure is completely absorbed.

10. The method of claim 1, further comprising monitoring tissue regeneration endoscopically.

11. A synthetic support structure comprising:
a body, the body having a first end and a second end opposed to the first end, the body further having a least one portion configured as a tubular member, the body comprising an outwardly oriented surface, the outwardly oriented surface having at least one region composed of spun polymeric fibers, the spun polymeric fibers having an average fiber diameter between 15 nm and 10 microns, at least a portion of the spun polymeric fibers interlinked to form pores having an average pore diameter less than 50 microns.

12. The synthetic support structure of claim 11 wherein the spun polymeric fibers are electrospun, are interconnected and form an outer layer of the body and the body further comprises at least one inner layer, the inner layer composed of at least one of a polymeric mesh, a polymeric braided support material, a solid polymeric member, an electrospun layer, the outer layer in overlying contact with the inner layer.

13. The synthetic support structure of claim 12 wherein the electrospun polymeric fibers have an average fiber diameter of 3 to 10 micrometers and is composed of at least one of one of the following polymeric materials: polyvinylidene fluoride, syndiotactic polystyrene, copolymer of vinylidene fluoride and hexafluoropropylene, polyvinyl alcohol, polyvinyl acetate, poly(acrylonitrile), copolymers of polyacrylonitrile and acrylic acid, copolymers of polyacrylonitrile and methacrylates, polystyrene, poly(vinyl chloride), copolymers of poly(vinyl chloride), poly(methyl methacrylate), copolymers of poly(methyl methacrylate), polyethylene terephthalate, polyurethane and wherein at least one layer is a polymeric material containing polyethylene terephthalate, polyurethane, blends of polyethylene terephthalate and polyurethane.

14. The synthetic support structure of claim 13 polymeric braided support material is composed of at least one of polyethylene terephthalate, polyurethane, nitinol and mixtures thereof.

15. The synthetic support structure of claim 14 further comprising at least one cellular sheath layer, the cellular sheath layer composed of cellular material, the cellular material composed of mesenchymal cells and stem cells present in a layer, the layer being between 1 and 100 celled thick. wherein the cellular sheath layer of cellular material overlay the electrospun polymeric materials present on the outer polymeric surface such that the cellular material is contained on the outer polymeric surface and spans pores defined therein.

16. A method of performing a surgical procedure in gastrointestinal tract of a subject having a cricopharyngeal notch and a suprasternal notch, the subject further having a diaphragm, and a stomach, the stomach having a fundus and fundal proximal region, the method comprising the steps of:
removing a circumferential portion of an esophagus from the subject forming a resection site, wherein the esophagus has a cervical esophagus region, wherein the circumferential portion of the esophagus to be removed is located between the cervical esophagus region and the fundus of the stomach forming a cervical-distal anastomosis and a fundal proximal anastomosis, wherein the cervical-distal anastomosis and/or the fundal proximal anastomosis define a native tissue lumenal surface;
replacing the removed circumferential portion with a synthetic support structure, the synthetic support structure having a first end, a second end opposed to the first end and middle section extending between the first end and the second end, wherein at least a portion of the synthetic support structure is configured as a tubular member defining an interior lumen, the synthetic support structure further having an outer polymeric surface extending from the first end to the second end, and a cellularized layer adhering to at least a portion of the outer polymeric surface;
maintaining the first end of the synthetic support structure in direct contact and sutured to distal cervical esophageal tissue creating a cervical-synthetic support anastomosis junction;
maintaining the second end of the synthetic support structure in direct contact and sutured to fundal proximal tissue creating a synthetic support-fundal anastomosis, wherein the first end and the second end are maintained in contact for a period of time sufficient to achieve neo-esophageal tissue growth along the synthetic structural support, the neo-esophageal tissue growth derived from and in contact with the tissue present in the resected organ remaining in the subject, the neo-esophageal tissue growth occurring around the synthetic tubular support at a location between the cricophayngeus anastomosis and the fundal proximal anastomosis of the subject; and
after achieving neoesophageal tissue growth, removing the synthetic support structure from contact with the esophagus in a manner such that the neo-esophageal tissue growth is continuous and remains in contact with a cervical-distal portion of the esophagus and a fundal proximal portion of the esophagus.

17. The method of claim 16 further comprising:
imparting cellular material onto the outer polymeric surface of the synthetic support structure; and
allowing the cellular material to grow to form the cellularized layer, the imparting and allowing steps occurring prior to replacing the removed circumferential portion with the synthetic support structure.

18. The method of claim 17 wherein the cellular material is derived from autologous stem cells harvested from the subject.

19. The method of claim 16, wherein at least a portion of the synthetic support structure is absorbed at the resection site within a period of time sufficient to achieve guided tissue growth along the synthetic support structure.

20. The method of claim 16 further comprising the step of positioning a first pressure member in the lumen of the synthetic support structure and lumenal surface of the native tissue after surgical placement of the synthetic support structure, wherein the first pressure member is one of a stent or a nasogastric tube.

21. The method of claim 20 further comprising the step of removing the first pressure member contemporaneous with removing the synthetic support structure or by active removal of the support structure using endoscopic techniques.

22. The method of claim 21 further comprising the step of positioning a second pressure member in the esophagus at a region defined by guided tissue grow at a time subsequent to the step of removing the first pressure member, the second pressure member remaining in position for an interval that allows epithelialization of the lumenal surface of the neoesophageal tissue, wherein the second pressure member is a stent or a nasogastric tube.

23. The method of claim 19 wherein guided tissue growth occurs between the cricophayngeus notch and the suprasternal notch.

24. The method of claim 21 further comprising the step of translocating the fundal proximal region of the stomach of the subject to a position above the diaphragm of the subject.

25. The method of claim 23 wherein guided tissue growth comprises epithelial tissue, smooth muscle tissue, vascular tissue and neuronal cellular proteins and wherein the synthetic support structure has an outer surface that includes electrospun polymeric fibers and wherein the guided tissue growth overlies the synthetic support structure without adhering to the outer polymeric surface of the synthetic support structure.

26. The method of claim 25 wherein the first pressure member spans at least one junction between at least one anastomosis and at least one end of the synthetic support structure when in position.

27. The method of claim 26 wherein the first pressure member spans a junction between the cervical-distal anastomosis and the first end of the synthetic support structure and a junction between the fundal proximal anastomosis and the second end of the synthetic support structure.

28. The method of claim 27 further comprising the step of removing the first pressure member after achieving guided tissue growth, wherein the first pressure member is removed prior to or contemporaneous to removal of the synthetic support structure.

29. The method of claim 28 further comprising the step of positioning a second pressure member in the esophagus at a region defined by guided tissue grow at a time subsequent to the step of removing the first pressure member, the second pressure member remaining in position for an interval of at least 15 days.

30. The method of claim 26 further comprising the step of translocating the fundal proximal anastomosis of the stomach of the subject to a position above the diaphragm of the subject.

31. The method of claim 30 further comprising:
imparting cellular material onto the outer polymeric surface of the synthetic support structure; and
allowing the cellular material to grow to form the cellularized layer, the imparting and allowing steps occurring prior to replacing the removed circumferential portion with the synthetic support structure.

32. The method of claim 31 wherein the cellular material is derived from stem cells autologously derived from the subject.

33. The method of claim 32 wherein the guided tissue growth occurs between the cricophayngeus and the suprasternal notch.

34. The method of claim 33 wherein the synthetic support structure comprises at least one region composed of electrospun polymeric fibers having an average fiber diameter between 15 nm and 10 microns, wherein at least a portion of the electrospun polymeric fibers are interlinked to form pores having an average pore diameter less than 50 microns, the pores defining at least one porous region present on the outer polymeric surface.

35. The method of claim 34 wherein the electrospun fibers of the synthetic support structure are interconnected and form an outer layer of the synthetic support structure and the synthetic support structure further comprises at least one inner layer, the inner layer composed of at least one of a polymeric mesh, a polymeric braided support material, a solid polymeric member, and an electrospun layer, the outer layer in overlying contact with the inner layer.

36. The method of claim 35 wherein the electrospun material of the synthetic support structure has an average fiber diameter of 3 to 10 micrometers wherein the electrospun material of the synthetic support structure is selected from the group consisting of polyethylene terephthalate, polyurethane, blends of polyethylene terephthalate and polyurethane and wherein cellular material present on the synthetic support structure overlays and adheres to the electrospun fibers present on the outer polymeric surface such that the cellular material, is contained on the outer polymeric surface and spans pores defined therein.
